(19) United States
(12) Patent Application Publication
(10) Pub. No.: US 2025/0387225 A1
(43) Pub. Date: Dec. 25, 2025

(54) PROSTHETIC MEDICAL DEVICE DELIVERY APPARATUS

(71) Applicant: EDWARDS LIFESCIENCES CORPORATION, Irvine, CA (US)

(72) Inventors: Kevin Gantz, Laguna Beach, CA (US); Tri D. Tran, Fountain Valley, CA (US)

(21) Appl. No.: 19/306,794

(22) Filed: Aug. 21, 2025

Related U.S. Application Data
(63) Continuation of application No. PCT/US2024/018313, filed on Mar. 4, 2024.
(60) Provisional application No. 63/488,511, filed on Mar. 5, 2023.

(51) Int. Cl. A61F 2/24

(52) U.S. Cl. CPC A61F 2/2409

(57) ABSTRACT

Devices and methods for delivery devices for docking devices are disclosed. As an example, a delivery apparatus can include a housing, a linear actuator coupled to the housing, the linear actuator comprising a traveler and an articulation member coupled to the traveler, wherein the traveler translates axially relative to the housing based on rotation of the articulation member relative to the housing, a first shaft extending through the housing and configured to translate axially relative to the housing, a second shaft extending through the first shaft and coupled to the traveler of the linear actuator such that the second shaft and the traveler translate axially together, and a locking mechanism coupled to the housing, wherein the first shaft is prevented from moving relative to the housing in a locked configuration, and wherein the first shaft is moveable relative to the housing in an unlocked configuration.

### CROSS REFERENCE TO RELATED APPLICATIONS

This application is a continuation of PCT patent application no. PCT/US2024/018313 filed on Mar. 4, 2024, which application claims the benefit of U.S. Provisional Patent Application No. 63/488,511, filed Mar. 5, 2023, each of these applications being incorporated herein in its entirety by this specific reference.

### FIELD

The present disclosure relates to delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery or femoral vein) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

In some examples, a docking device can be implanted first within the native valve and can be configured to receive a prosthetic valve and secure (e.g., anchor) the prosthetic valve in a desired position within the native valve. For example, the docking device can form a more circular and/or stable anchoring site at the native valve annulus in which a prosthetic valve can be expanded and implanted. A transcatheter delivery apparatus can be used to deliver the docking device to the implantation site.

### SUMMARY

Described herein are prosthetic heart valves, docking devices, delivery apparatuses, and methods for implanting prosthetic heart valves. The disclosed prosthetic heart valves, docking devices, delivery apparatuses, and methods can, for example, provide improved positioning of a docking device by independent actuation of multiple shafts of a delivery apparatus. As such, the devices and methods disclosed herein can, among other things, overcome one or more of the deficiencies of typical prosthetic heart valves, docking devices and associated delivery apparatuses.

A delivery apparatus can comprise a handle and one or more shafts coupled to the handle.

In some examples, a delivery apparatus can comprise a handle, a shaft coupled to the handle, and a linear actuator coupled to the shaft, wherein the linear actuator is configured to move the shaft in an axial direction relative to the handle.

In some examples, a delivery apparatus can comprise a housing; a linear actuator coupled to the housing, the linear actuator comprising a traveler and an articulation member coupled to the traveler, wherein the traveler translates axially relative to the housing based on rotation of the articulation member relative to the housing; a first shaft extending through the housing and configured to translate axially relative to the housing; a second shaft extending through the first shaft and coupled to the traveler of the linear actuator such that the second shaft and the traveler translate axially together; and a locking mechanism coupled to the housing, wherein the first shaft is prevented from moving relative to the housing in a locked configuration, and wherein the first shaft is moveable relative to the housing in an unlocked configuration.

In some examples, a delivery apparatus can comprise a housing including an opening at a distal end; a first shaft extending through the housing and configured to translate relative to the housing; a second shaft extending through the first shaft; and a linear actuator coupled to the housing, the linear actuator comprising a traveler and an articulation member coupled to the traveler, wherein the traveler translates axially relative to the housing between a first axial position and a second axial position based on rotation of the articulation member relative to the housing, wherein the second shaft and the traveler are configured to translate together, and wherein the traveler extends at least partially out of the opening of the housing in the second axial position.

In some examples, a delivery apparatus can comprise a housing defining an interior region; a linear actuator coupled to the housing and configured to translate axially relative to the housing, the linear actuator comprising a lead screw and a chassis coupled to the lead screw, the chassis positioned within the interior region of the housing; a pusher shaft extending through the housing and coupled to the lead screw, wherein the pusher shaft and the lead screw are configured to translate together relative to the housing; and a cap coupled to the housing and at least partially removable from the housing to selectively expose the interior region.

In some examples, a delivery apparatus comprises a housing; a sleeve shaft extending through the housing, wherein the sleeve shaft comprises a u-shaped or c-shaped axial cross section; and a locking mechanism coupled to the housing and comprising a collet having a lumen, wherein the sleeve shaft extends through the lumen, wherein the lumen comprises a non-circular cross-section, wherein the sleeve shaft is prevented from moving relative to the housing in a locked configuration, and wherein the sleeve shaft is moveable relative to the housing in an unlocked configuration.

A method for implanting a prosthetic medical device at a target implantation site can comprise moving a pusher shaft of a delivery apparatus in an axial direction relative to a sleeve shaft and a hub assembly of the delivery apparatus.

In some examples, a method for implanting a prosthetic medical device at a target implantation site in a subject, the method comprising: advancing a prosthetic medical device coupled to a distal end of a pusher shaft of a delivery apparatus and retained within a sleeve shaft of the delivery apparatus towards the target implantation site by moving the sleeve shaft and the pusher shaft in a distal direction relative to a handle of the delivery apparatus; locking a position of the sleeve shaft relative to a hub assembly of the delivery apparatus with a locking mechanism coupled to the hub assembly of the delivery apparatus; and actuating a linear actuator of the hub assembly to move the pusher shaft in an axial direction relative to the sleeve shaft and the hub assembly.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 schematically illustrates a stage in a docking device implantation procedure where a guide catheter is inserted into a blood vessel of a patient and navigated through the blood vessel and into a heart of the patient, according to one example.

FIG. 6 schematically illustrates another stage in the example docking device implantation procedure where a distal end portion of a docking device delivery apparatus is advanced from the guide catheter and into a left ventricle of the heart.

FIG. 7 schematically illustrates another stage in the example docking device implantation procedure where the distal end portion of the docking device delivery apparatus is coiled around a plurality of leaflets of the heart.

FIG. 8 schematically illustrates another stage in the example docking device implantation procedure where a radius of curvature of the distal end portion of the docking device delivery apparatus is increased to encircle the chordae tendineae of the heart in a variable encircling turn.

FIG. 9 schematically illustrates another stage in the example docking device implantation procedure where a sleeve shaft of the docking device delivery apparatus is retracted in a proximal direction to unsheathe a guard member of a docking device.

FIG. 10 schematically illustrates another stage in the example docking device implantation procedure where the sleeve shaft is advanced in the distal direction to foreshorten the guard member.

FIG. 11 schematically illustrates another stage in the example mitral valve replacement procedure where the docking device delivery apparatus is decoupled from the docking device.

FIG. 12 is a side view of a delivery apparatus for a docking device, according to one example.

FIG. 13 is a side view of a dock handle of the delivery apparatus of FIG. 12.

FIG. 14 is a perspective view of the dock handle of FIG. 13, with a cover partially removed from the handle.

FIG. 15 is a perspective view of the dock handle of FIG. 13, with the cover omitted for purposes of illustration.

FIG. 16 is a cross-sectional side view of the dock handle of FIG. 13.

FIGS. 17A-17C are perspective views of the dock handle of FIG. 13 in different configurations.

FIG. 18 is a perspective view of a collet of a locking mechanism, according to one example.

FIG. 19A is an end view of a collet of a locking mechanism, according to one example.

FIG. 19B is a perspective view of the collet of FIG. 19A.

FIG. 20 is a perspective view of a docking device for use with a docking device delivery apparatus, according to one example.

FIG. 21 is a perspective view of shafts of the delivery apparatus of FIG. 12.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Disclosed herein are examples of a delivery system that can be used to navigate a subject's vasculature to deliver a prosthetic medical device (such as a docking device used in conjunction with a prosthetic heart valve), tools, agents, or other therapy to a target implantation site within the body of the subject. In connection therewith, delivery devices described herein, in some examples, can include a plurality of shafts that are independently actuated relative to one another to improve the positioning of the prosthetic medical device within the body of the subject. Disclosed herein are exemplary devices and/or methods that can, among other things, make it easier to actuate (e.g., axially move) one or more components of a delivery device relative to one or more other components of the delivery device.

### Examples of the Disclosed Technology

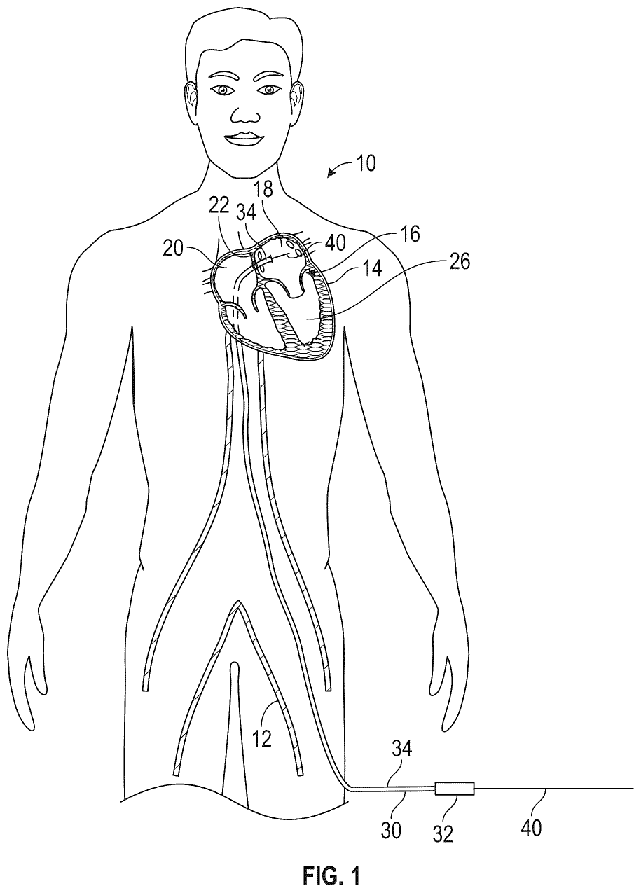

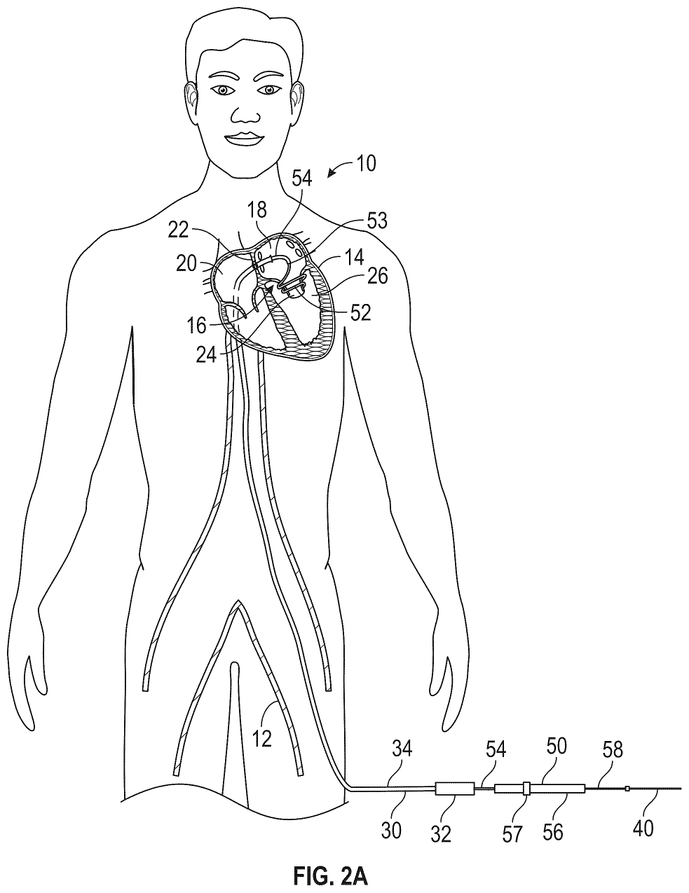

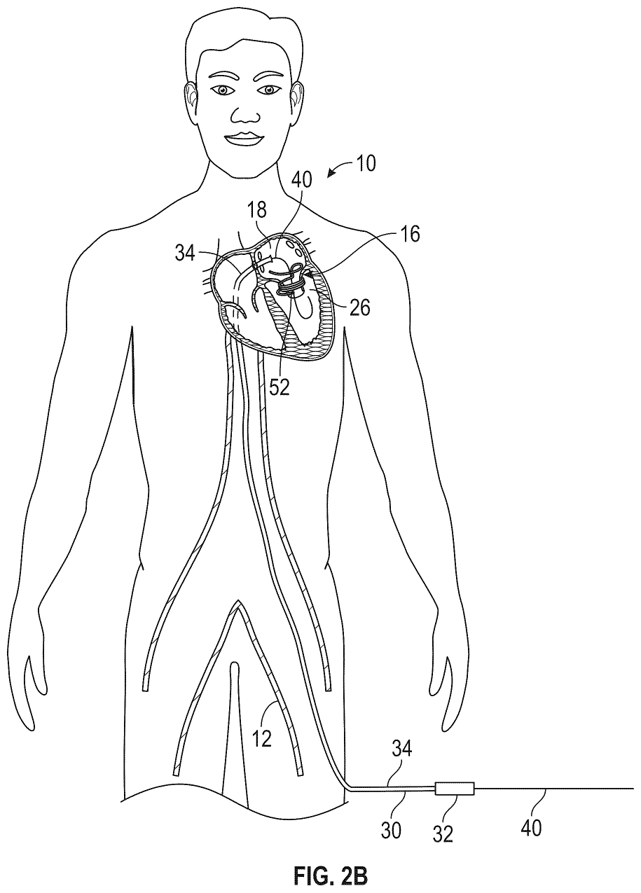

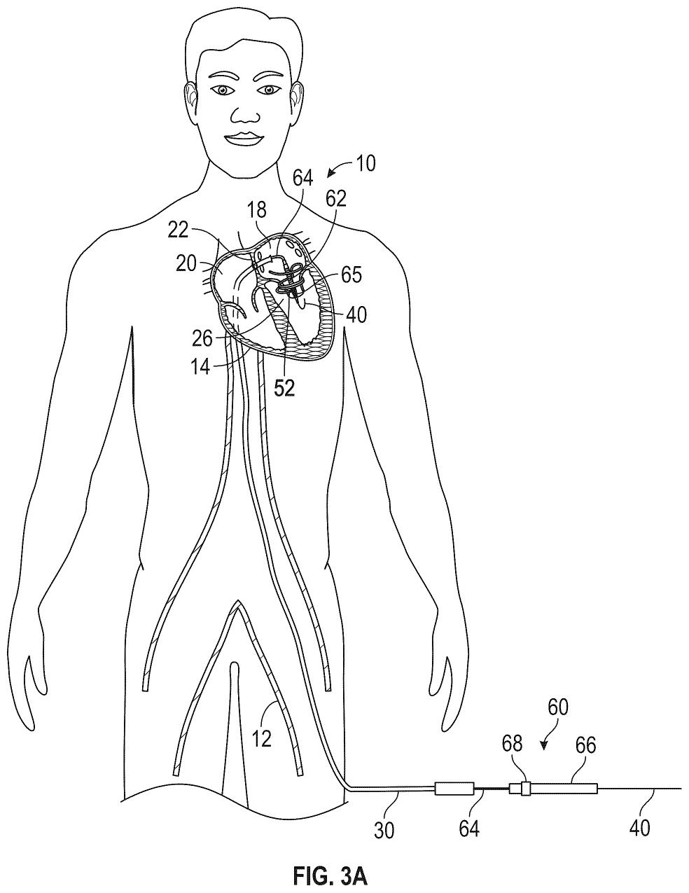

FIGS. 1-4 depict an example of a transcatheter heart valve replacement procedure (such as a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a delivery apparatus 50 (FIG. 2A) and then removes the delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the delivery apparatus 50 and the prosthetic valve delivery apparatus 60 to be navigated through and along, to the implantation site (the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (such as the delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 (which may also be referred to as a catheter shaft 34) extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (such as the guide catheter 30, the delivery apparatus 50, the prosthetic valve delivery apparatus 60, additional catheters, or the like) and their associated devices (such as docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle 26 of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (such as through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a delivery apparatus 50 (which may also be referred to as an “implant catheter,” a dock delivery system,” a “docking device delivery apparatus,” and/or a “docking device delivery device”).

In general, the delivery apparatus 50 comprises a delivery shaft 54 (which may also be referred to as a “dock delivery system shaft”), a handle 56 (which may also be referred to as a “dock delivery system handle”), and a pusher assembly 58. The delivery shaft 54 is configured to be advanced through the patient's vasculature (blood vessel 12) and to the implantation site (such as native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the delivery apparatus 50 is configured to be gripped and/or otherwise held by the user, outside the body of the patient 10, to advance the delivery shaft 54 through the patient's vasculature (such as the blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (such as the native mitral valve 16). For example, the pusher assembly 58 is configured to be adjusted by the user to push the docking device 52 out of the distal end portion 53 of the delivery shaft 54. A shaft (which may also be referred to as a “pusher shaft”) of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the delivery apparatus 50 such that the docking device 52 can be released after being deployed at the native mitral valve 16.

Further details of the docking device delivery apparatus and its variants are described in International Publication Nos. WO2020/247907 and WO2023/205076 and International Application No. PCT/US2023/033745, which are incorporated by reference herein in their entirety.

Referring again to FIG. 2A, after the guide catheter 30 is positioned within the left atrium 18, the user may insert the delivery apparatus 50 (such as the delivery shaft 54) into the patient 10 by advancing the delivery shaft 54 of the delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the delivery apparatus 50 through the blood vessel 12 along the guidewire 40 until the delivery shaft 54 reaches the left atrium 18, as illustrated in FIG. 2A. Specifically, the user may advance the delivery shaft 54 of the delivery apparatus 50 by gripping and exerting a force on (for example, by pushing) the handle 56 of the delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

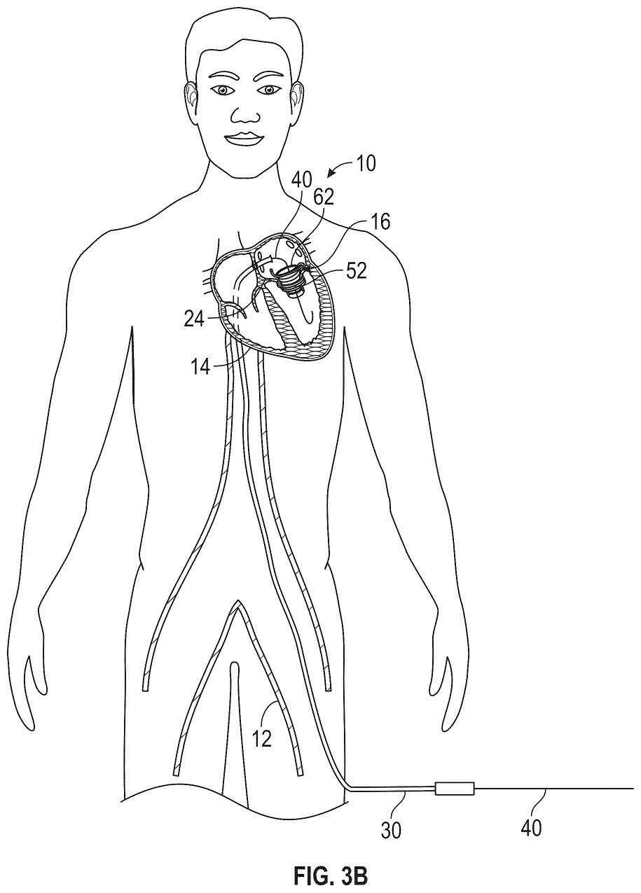

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (such as the articulation members 57). In some examples, the user can fine tune the positioning of the distal end portion 53 of the delivery shaft 54 using the procedure described below in connection with FIGS. 5-11. After the delivery shaft 54 is positioned, the user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (such as the portion of the docking device 52 shown in FIG. 2A that is configured to be positioned within the left ventricle 26 and/or on the ventricular side of the native mitral valve 16), the user may then deploy the remaining portion of the docking device 52 (such as an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the delivery apparatus 50 from the docking device 52. Once the docking device 52 is disconnected from the delivery apparatus 50, the user may retract the delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the delivery apparatus 50 (including the delivery shaft 54) has been removed from the patient 10 such that only the guidewire 40 and the guide catheter 30 remain inside the patient 10. In some examples, after removing the delivery apparatus 50, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (such as the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, by pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, by inflating the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (such as tricuspid, pulmonary, and/or aortic valves). Further, the same and/or similar delivery apparatuses (such as the delivery apparatus 50, prosthetic valve delivery apparatus 60, guide catheter 30, and/or guidewire 40), docking devices (such as the docking device 52), replacement heart valves (such as the prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

FIGS. 5-11 schematically illustrate a procedure for implanting a prosthetic medical device at a target implantation site in a subject (such as the patient 10). In some examples, the procedure is a docking device implantation procedure for implanting a docking device 152 at an annulus of the native mitral valve 16 of the patient 10. In some examples, one exemplary difference between the docking device 52 of FIGS. 1-4 and the docking device 152 of FIGS. 5-11 can be that the docking device 152 optionally includes a guard member 180 coupled to the docking device 152, wherein the guard member 180 can be configured to further mitigate the possibility of paravalvular leakage between the annulus of the native mitral valve 16 and a prosthetic heart valve (such as the prosthetic heart valve 62) positioned in the docking device 152.

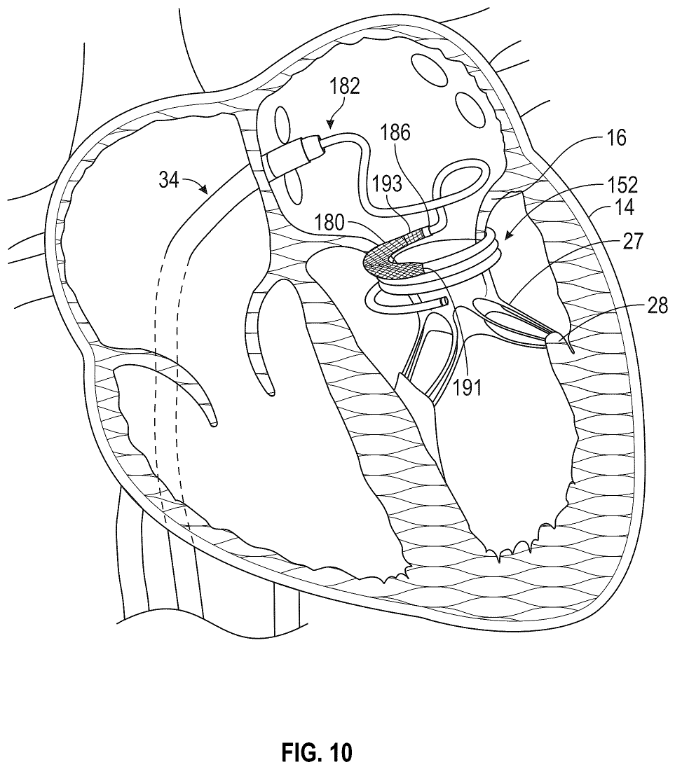

The procedure of FIGS. 5-11 can be performed using a delivery apparatus 150 (which may also be referred to as a “docking device delivery apparatus”). In some examples, the delivery apparatus 150 can be used as the docking device delivery apparatus 50 in a prosthetic valve implantation procedure, as described above with reference to FIGS. 1-4. The delivery apparatus 150 can comprise three independently actuatable shafts: a delivery shaft 154 (which may also be referred to as a “dock delivery system shaft”), a sleeve shaft 182, and a pusher shaft 184 (which may also be referred to as a “dock shaft”) (FIG. 12). The pusher shaft 184 can be disposed within the sleeve shaft 182, which can in turn be disposed within the delivery shaft 154. In some examples, the delivery shaft 154, the sleeve shaft 182, and the pusher shaft 184 can be coaxial. The delivery shaft 154, the sleeve shaft 182, and the pusher shaft 184 can be independently actuated relative to one another in the axial direction during the docking device implantation procedure to better position the docking device 152 within the annulus of native mitral valve 16, such that the implanted docking device 152 can better encircle one or more chordae tendineae 27 of the heart 14 and provide for better sealing between the implantation site and a prosthetic heart valve (such as the prosthetic heart valve 62).

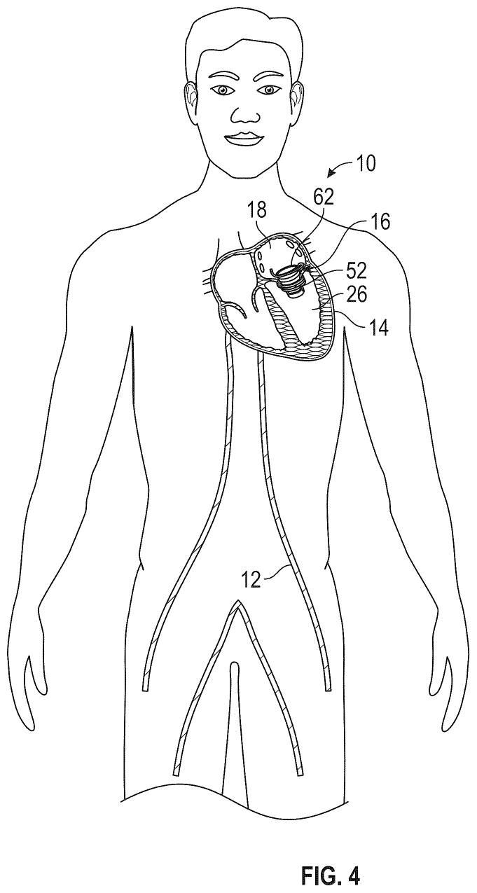

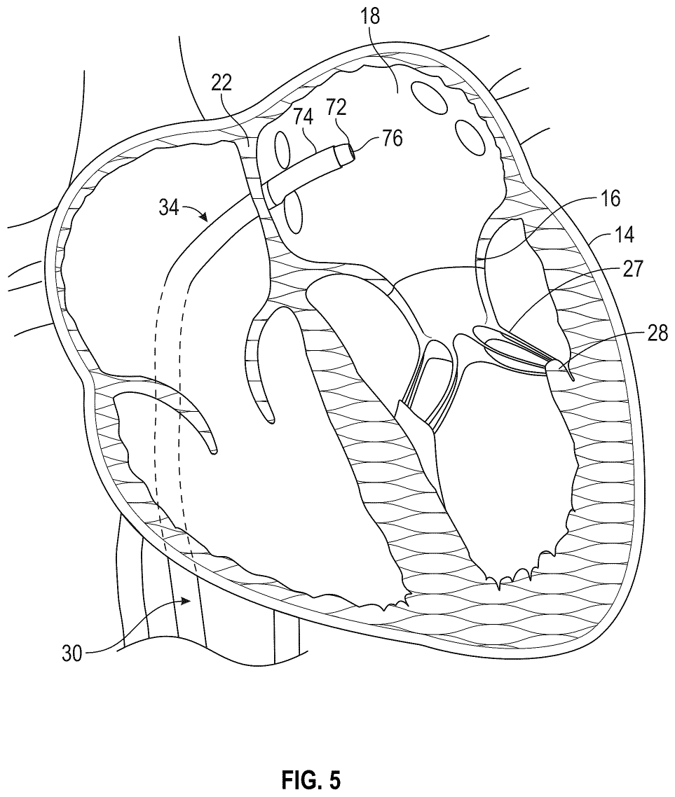

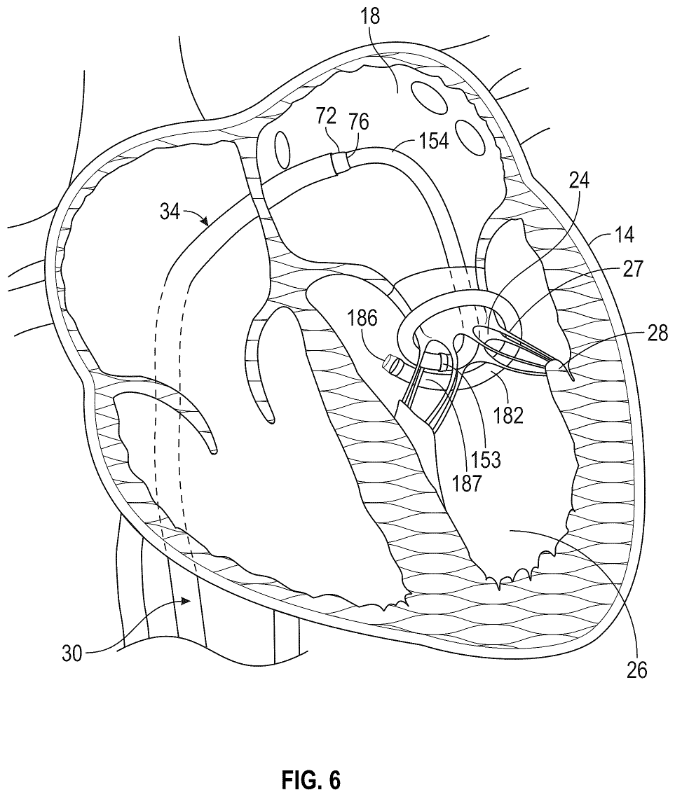

During the procedure, a user of the delivery apparatus 150 first creates a pathway to a patient's native heart valve using the guide catheter 30 (FIG. 5). The user then distally advances a distal end portion of the delivery apparatus 150 to advance the docking device 152 to the target implantation site (FIG. 6-7). In some examples of the procedure, the user can actuate the delivery system to change or adjust the curvature of the distal end portion of the delivery apparatus 150 (see, e.g., the leading turn 187 of the delivery apparatus 150 in FIGS. 7-8). This adjustable radius of curvature can be referred to as a “variable encircling turn” (VET). The VET can, for example, make it easier to encircle one or more chordae tendineae 27 connecting the leaflets 24 to the papillary muscles 28 of the heart 14 by proximally retracting the pusher shaft 184 relative to the sleeve shaft 182.

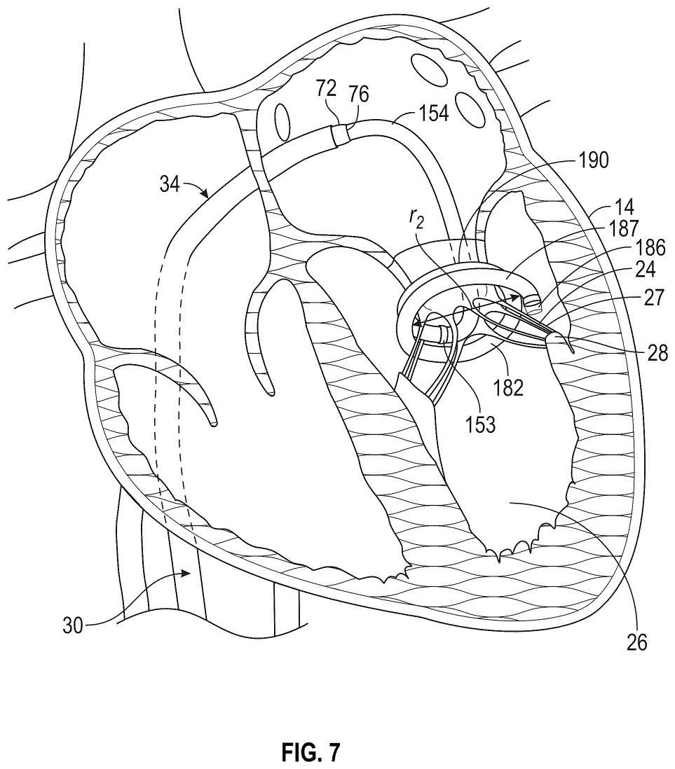

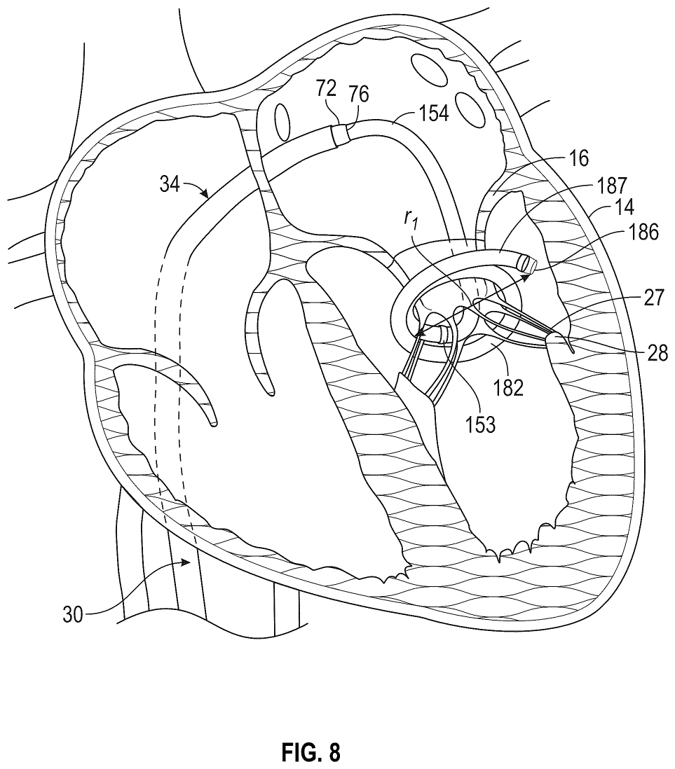

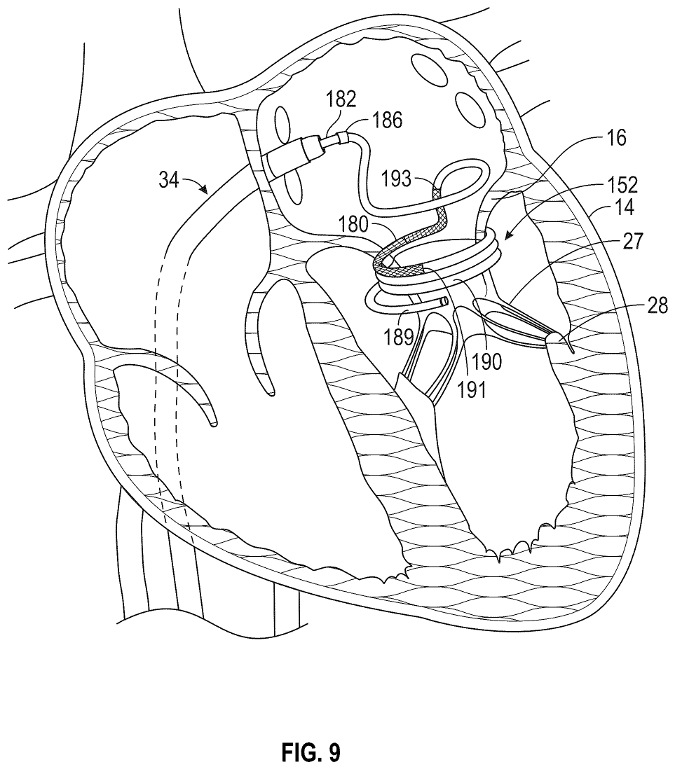

In some examples where the docking device 152 further comprises a guard member 180, the user can then retract the delivery shaft 154 and the sleeve shaft 182 in the proximal direction to expose the guard member 180 (FIG. 9) from the sleeve shaft 182. In some examples, the user can then advance the sleeve shaft 182 in the distal direction to exert an axially compressive force against the guard member 180, thereby axially foreshortening and radially expanding the guard member 180 (FIG. 10). Finally, the user can decouple the docking device 152 from the pusher shaft 184 and remove the delivery apparatus 150 from the patient 10 (FIG. 11).

FIG. 5 illustrates a stage in the procedure in which the guide catheter 30 is advanced in a distal direction through the patient's vasculature and into the left atrium 18 of the heart 14. The guide catheter 30 comprises the catheter shaft 34 that includes a distal end 72, a flex region 74, and a lumen exit 76 on the distal end 72 of the catheter shaft 34. The lumen exit 76 is connected to a catheter shaft lumen disposed within the catheter shaft 34. A delivery apparatus (such as any of the prosthetic device delivery apparatuses or implant catheters described herein) is configured to be disposed within the catheter shaft lumen. The catheter shaft lumen extends from a proximal end portion of the catheter shaft 34 (such as the portion of the catheter shaft 34 coupled to the handle 32) to the lumen exit 76. The guide catheter 30 is positioned such that the distal end 72 of the catheter shaft 34 is disposed within the left atrium 18 of the heart 14.

In some examples, the catheter shaft 34 can comprise one or more pull wires for adjusting a curvature of the flex region 74 of the catheter shaft 34. In some examples, the pull wires can extend through a lumen coupled to the lumen exit 76 and can couple to a portion of the catheter shaft 34, such as a pull wire ring at or adjacent the distal end 72. In some examples, the pull wires can extend through one or more pull wire lumens embedded in the catheter shaft 34. In some examples, adjusting a tension of the pull wires can adjust the curvature of a flex region 74 of the catheter shaft 34. In some instances, the catheter shaft 34 (including its flex region 74) can be integrally formed as a single, unitary component. In some instances, the catheter shaft 34 can comprise one or more segments (for example, the flex region 74, other regions, etc.) that are formed as separate components that are coupled together (such as via fasteners, adhesive, mating features, and/or other means for coupling). In some examples, the flex region 74 can comprise a material that is more prone to flexing, bending, twisting, etc. than the remaining portion of the catheter shaft 34 (for example, a polymer having relatively lower durometer hardness). This can enable the curvature of the flex region 74 to be adjusted or increased at a different rate than the remaining portion of the catheter shaft 34 when the pull wires are tensioned. For example, the curvature of the flex region 74 can change at an increased rate relative to the proximal portion of the catheter shaft 34 as the tension of the pull wires is increased. The catheter shaft 34 can also include one or more reinforcing braids or jackets that makes the catheter shaft 34 more resistant to flexing, bending, twisting, etc., for example, to prevent one or more of the lumens from kinking or collapsing when the catheter shaft 34 is manipulated.

During this stage, the docking device 152 is disposed within the sleeve shaft 182, which in turn is disposed within the delivery shaft 154, which in turn is disposed within the catheter shaft 34. The pusher shaft 184 is disposed proximally adjacent the docking device 152 within the sleeve shaft 182. In some examples, the docking device 152, the sleeve shaft 172, the delivery shaft 154, and the catheter shaft 34 can be coaxially aligned. During this stage, the docking device 152 is in a generally straight delivery configuration (i.e., without any coiled or looped portions, but can be flexed or bent) so as to maintain a small radial profile when moving through a patient's vasculature.

FIG. 6 illustrates a stage in the procedure in which the docking device 152, the delivery shaft 154, the sleeve shaft 182, and the pusher shaft 184 are advanced in a distal direction through the lumen exit 76 of the catheter shaft 34, through the left atrium 18, and to the native mitral valve 16. The docking device 152 is disposed within a sleeve shaft lumen of the sleeve shaft 182, which in turn is disposed within a delivery shaft lumen of the delivery shaft 154. The pusher shaft 184 is disposed proximally adjacent the docking device 152 within the sleeve shaft 182.

The delivery shaft 154, which in some examples can resemble the delivery shaft 54, comprises the delivery shaft lumen through which the sleeve shaft 182 and the pusher shaft 184 can extend. The delivery shaft lumen is configured to extend in the axial direction along the length of the delivery shaft 154 between a handle of the delivery apparatus 150 and a distal end portion 153 of the delivery shaft 154. The sleeve shaft 182 and the pusher shaft 184 are configured to exit the delivery shaft lumen through an opening at the distal end portion 153.

The sleeve shaft 182 is configured to extend through the delivery shaft 154 and sheathe the docking device 152 and at least a portion of the pusher shaft 184 as the docking device 152 is navigated through the patient's vasculature to the native mitral valve 16. The sleeve shaft 182 comprises the sleeve shaft lumen extending along the length of the sleeve shaft 182 between a handle of the delivery apparatus 150 and a distal end portion 186 of the sleeve shaft 182. In some examples, a portion (e.g., a proximal end portion) of the sleeve shaft 182 can have a substantially U-shaped axial cross-section or other shape that allows the proximal end portion of the pusher shaft 184 to exit the sleeve shaft 182 at a location that is distal to the proximal end of the sleeve shaft 182. The distal end portion of the pusher shaft 184 can exit the sleeve shaft 182 at an opening at the distal end portion 186 of the sleeve shaft 182.

The distal end portion 186 of the sleeve shaft 182 is configured to capture the native tissue (e.g., the native leaflets 24 and chordae 27). The sleeve shaft 182 can have a relatively low-friction and/or lubricious outer surface to reduce the likelihood of the sleeve shaft 182 snagging on the native tissue.

In some examples, the sleeve shaft 182 can comprise a plurality of layers. For example, the sleeve shaft 182 can comprise an inner-most polymeric layer, a braided or other type of flexible reinforcing layer, and an outer-most polymeric layer. In some examples, the reinforcing layer is a shape memory material and/or elastic material (e.g., nitinol and/or stainless steel).

In some instances, the distal end portion 186 of the sleeve shaft 182 can be curved to help facilitate the capture of the native tissue. This can be accomplished by forming the distal end portion 186 of the sleeve shaft 182 in a curved configuration and/or by forming the sleeve shaft 182 of a relatively more flexible material than the docking device 152 and advancing the curved docking device 152 into the sleeve shaft 182, which can result in the sleeve shaft 182 assuming a curved configuration and/or the curvature of the sleeve shaft 182 being altered by the docking device 152.

In this manner, the distal end portion 186 of the sleeve shaft 182 can form a sleeve shaft leading turn 187 configured to capture the chordae tendineae 27 as the sleeve shaft 182 is advanced around the leaflets 24 of the native mitral valve 16. The sleeve shaft leading turn 187 is a portion of the sleeve shaft 182 disposed at or adjacent the distal end portion 186 that comprises a curved portion of the sleeve shaft 182 having a radius of curvature. When the docking device 152 is not sheathed within the portion of the sleeve shaft 182 corresponding to the sleeve shaft leading turn 187, the sleeve shaft leading turn 187 has a radius of curvature equal to a first radius of curvature (r₁) As discussed later in this application, particularly in reference to FIGS. 7-8, the radius of curvature of the sleeve shaft leading turn 187 can be varied by relative movement between the sleeve shaft 182 and the docking device 152. In some examples, in which the sleeve shaft 182 can be constructed from, formed of, and/or comprise a shape memory material, the sleeve shaft 182 may originally be formed such that the sleeve shaft leading turn 187 has the first radius of curvature (r₁). The sleeve shaft leading turn 187 can be forced into another configuration having another radius of curvature (for example, second radius of curvature (r₂)) but can revert to its original configuration having the first radius of curvature (r₁) when the force is removed. In some examples, the second radius of curvature (r₂) can be less than the first radius of curvature (r₁).

In some examples, the sleeve shaft leading turn 187 can conform to a shape or curvature of another component (such as the docking device 152) sheathed by the sleeve shaft leading turn 187, such that the radius of curvature of the sleeve shaft leading turn 187 is equal to a corresponding radius of curvature of the other component. As such, the distal end portion 186 of the sleeve shaft 182 can have a lesser radius of curvature when a distal end portion (such as leading turn 189) of the docking device 152 is disposed at or proximate to the distal end portion 186 of the sleeve shaft 182. This is because the docking device 152 can have a smaller radius of curvature and can be relatively more rigid than the sleeve shaft 182. In some examples, the radius of curvature of the distal end portion 186 of the sleeve shaft 182 can be increased by moving the distal end of the docking device 152 proximally relative to the distal end portion 186 of the sleeve shaft 182 such that the sleeve shaft 182 can assume its pre-set configuration. This can be done by moving the docking device 152 proximally while maintaining the position of the sleeve shaft 182, by moving the sleeve shaft 182 distally relative to the docking device 152, or a combination of the two.

The pusher shaft 184 is configured to extend through the delivery shaft 154 and the sleeve shaft 182. The pusher shaft 184 is configured to be disposed proximally adjacent the docking device 152 within the sleeve shaft 182 while the docking device 152 is navigated through the patient's vasculature to the native mitral valve 16. When the pusher shaft 184 is moved in the axial direction relative to the sleeve shaft 182, the pusher shaft 184 can exert a force on the docking device 152 to move the docking device 152 in the axial direction. In some examples, the docking device 152 can be releasably coupled to the pusher shaft 184 via a connection mechanism of the delivery apparatus 150 such that the docking device 152 can be released after being deployed at the native mitral valve 16.

In some examples, the distal end portion 153 of the delivery shaft 154 can be positioned during this stage between the leaflets 24 of the native mitral valve 16 (such as at or near the posteromedial commissure). In some examples, the distal end portion 153 of the delivery shaft 154 can extend distally past the native mitral valve 16 and be positioned adjacent the native mitral valve 16 in the left ventricle 26. In some examples, the distal end portion 153 of the delivery shaft 154 can be positioned adjacent the native mitral valve 16 in the left atrium 18.

Once the distal end portion 153 of the delivery shaft 154 is positioned, the docking device 152, the sleeve shaft 182, and the pusher shaft 184 are advanced in a distal direction out of the opening at the distal end portion 153 of the delivery shaft 154, through the native mitral valve 16, and into the left ventricle 26.

FIG. 7 illustrates a stage in the procedure in which the docking device 152 (disposed within the sleeve shaft 182), the sleeve shaft 182, and the pusher shaft 184 (disposed within the sleeve shaft 182 and proximally adjacent the docking device 152) wrap around or encircle the leaflets 24 on the ventricular side of the native mitral valve 16. When the docking device 152 exits the delivery shaft 154, the docking device 152 assumes a coiled configuration that is configured to wrap around or encircle the leaflets 24 on the ventricular side of the native mitral valve 16. In some examples, in which the docking device 152 can be constructed from, formed of, and/or comprise a shape memory material, the docking device 152 may originally be formed in the coiled configuration, but may be forced into a straightened delivery configuration by the delivery shaft 154. The docking device 152 can assume its original coiled configuration once the docking device 152 is no longer sheathed by the delivery shaft 154.

As previously discussed, the portions of the sleeve shaft 182 that sheathe the docking device 152 can conform to or assume the shape and/or curvature of corresponding portions of the docking device 152. For example, the sleeve shaft leading turn 187 can conform to the leading turn 189 of the docking device 152, wherein the leading turn 189 has radius of curvature equal to the second radius of curvature (r₂). Thus, the sleeve shaft leading turn 187 can assume a configuration having the second radius of curvature (r₂). In other words, the variable encircling turn can be equal to the second radius of curvature (r₂).

FIG. 8 illustrates an optional stage in the procedure in which the radius of curvature of the sleeve shaft leading turn 187 (in other words, the variable encircling turn) is increased from the second radius of curvature (r₂) to the first radius of curvature (r₁) to better capture the chordae tendineae 27 within the docking device leading turn 189. In some examples, the radius of curvature of the sleeve shaft leading turn 187 can be increased by retracting the pusher shaft 184 in the proximal direction relative to the sleeve shaft 182, such that docking device leading turn 189 and/or the docking device 152 is no longer sheathed by the sleeve shaft leading turn 187. In some examples, the radius of curvature of the sleeve shaft leading turn 187 can be increased by advancing the distal end portion 186 of the sleeve shaft 182 in the distal direction relative to the docking device 152. When the sleeve shaft leading turn 187 is no longer forced to conform to the curvature of the docking device leading turn 189 having the second radius of curvature (r₂), the sleeve shaft leading turn 187 can revert to its original configuration having the first radius of curvature (r₁), which is larger than the second radius of curvature (r₂). Since the chordae tendineae 27 are captured within the sleeve shaft leading turn 187, increasing the variable encircling turn to the larger first radius of curvature (r₁) beneficially allows for more portions of the chordae tendineae 27 to be captured by the sleeve shaft leading turn 187 as it is advanced around the leaflets 24.

During the step illustrated in FIG. 8, the delivery shaft 154 can be kept stationary to preserve the position of the distal end portion 153 of the delivery shaft 154 relative to the native mitral valve 16 (such as at or near the posteromedial commissure). In some examples, the sleeve shaft 182 can be kept stationary to preserve the encircling position and/or radial orientation of the sleeve shaft 182 relative to the native mitral valve 16. In some examples, the docking device 152 and/or the pusher shaft 184 can be kept stationary while the sleeve shaft 182 is moved during this step. In some examples, neither the sleeve shaft nor the pusher shaft 184 are kept stationary during this step.

As shown in FIG. 8, the variable encircling turn can be adjusted after the sleeve shaft 182 has made one helical turn around the leaflets 24. However, in some examples, the variable encircling turn can be adjusted after the sleeve shaft 182 has formed a plurality of helical turns around the leaflets 24. In some examples, the variable encircling turn can be adjusted before any helical turns have been formed around the leaflets 24.

FIG. 9 illustrates an optional stage in the procedure in which the delivery shaft 154 and the sleeve shaft 182 are retracted in the proximal direction to unsheathe the guard member 180. The docking device 152 comprises a coil 188 that defines a central region 190 comprising a plurality of helical turns wrapped around the leaflets 24 and a docking device leading turn 189 extending from a distal end portion of the central region 190.

The docking device 152 can further comprise the guard member 180 disposed on the docking device 152 such that the guard member 180 is positioned at or near the native mitral valve 16 (such as at or near the posteromedial commissure) when the docking device 152 is implanted at the native mitral valve 16. In some examples, the guard member 180 can be disposed proximally adjacent a central region (FIG. 22), wherein the central region can comprise a plurality of helical turns when the docking device 152 is wrapped around the leaflets 24. The guard member 180 can extend between a distal end portion 191 that is fixedly coupled to the docking device 152 and a movable proximal end portion 193 that can be moved along at least a portion of the docking device 152 in the axial direction. In some examples, the distal end portion 191 of the guard member 180 can abut the central region 190.

During the stages of the docking device implantation procedure shown in FIGS. 5-8, the guard member 180 can be covered by the delivery shaft 154 and the sleeve shaft 182. However, during the stage illustrated in FIG. 9, relative movement between the delivery shaft 154, the sleeve shaft 182, and the pusher shaft 184 can unsheathe the guard member 180. In some examples, the sleeve shaft 182 can be retracted in the proximal direction from the left ventricle 26, through the mitral valve 16, and into the left atrium 18 such that the distal end portion 186 of the sleeve shaft 182 is proximally closer to the user than the proximal end portion 193 of the guard member 180. In some examples, the distal end portion 186 of the sleeve shaft 182 can be distally disposed relative to the lumen exit 76. In some examples, the guard member 180 can be unsheathed by advancing the pusher shaft 184 distally relative to the sleeve shaft 182.

In some examples, the delivery shaft 154 can be retracted through the left atrium 18 in the proximal direction such that the distal end portion 153 of the delivery shaft 154 is proximally closer to the user than the proximal end portion 193 of the guard member 180. In some examples, the delivery shaft 154 can be retracted through the lumen exit 76 and into the catheter shaft lumen of the catheter shaft 34. In some examples, the pusher shaft 184 can be advanced distally relative to the delivery shaft 154 such that the distal end portion 153 of the delivery shaft 154 is proximally disposed relative to the guard member 180.

FIG. 10 illustrates an optional “seating” stage in the procedure in which the sleeve shaft 182 is distally advanced relative to the docking device 152 to axially foreshorten and radially expand the guard member 180. In some examples, the sleeve shaft 182 can be advanced in the distal direction such that the distal end portion 186 of the sleeve shaft 182 abuts and contacts the proximal end portion 193 of the guard member 180. In some examples, the pusher shaft 184 (and the docking device 152 coupled to the pusher shaft 184) can be retracted in the proximal direction such that the distal end portion 186 of the sleeve shaft 182 abuts and contacts the proximal end portion 193 of the guard member 180. The sleeve shaft 182 exerts a force upon the guard member 180 to distally advance the proximal end portion 193 of the guard member 180 relative to the docking device 152. Since the distal end portion 191 of the guard member 180 is fixedly coupled to the docking device 152, exerting the force upon the guard member 180 axially foreshortens and radially expands the guard member 180 to a deployed configuration. When in the deployed configuration, the guard member 180 further reduces the possibility of paravalvular leakage between the native mitral valve 16 and a prosthetic heart valve (such as the prosthetic heart valve 62). The frictional engagement between the proximal end of the guard member 180 and the docking device 152 can retain the position of the guard member 180 relative to the docking device 152 when the sleeve shaft 182 is retracted from the proximal end of the guard member 180.

FIG. 11 illustrates a stage in the procedure in which the delivery apparatus 150, including the delivery shaft 154 and the sleeve shaft 182, are retracted through the catheter shaft lumen of the catheter shaft 34. In some examples, the docking device 152 can be connected to the pusher shaft 184 via a release suture 194 that can be configured to be tied to the docking device 152. The release suture 194 can be cut during this stage to release the docking device 152 from the delivery apparatus 150.

FIG. 12 illustrates the delivery apparatus 150, according to one example. The delivery apparatus 150 can also be referred to as a “dock delivery apparatus,” “dock delivery catheter,” or “dock delivery system.” The delivery apparatus 150 comprises the delivery shaft 154, a handle 156 (which may also be referred to as a “dock delivery system handle”) coupled to a proximal end portion of the delivery shaft 154, a sleeve shaft 182 configured to extend through the delivery shaft 154 and the handle 156, a hub assembly 200 (which may also be referred to as a “dock handle”) coupled to a proximal end portion of the sleeve shaft 182, the pusher shaft 184 configured to extend through the handle 156 and the sleeve shaft 182, and a sleeve handle 196 coupled to a proximal end portion of the sleeve shaft 182.

The delivery shaft 154, which in some examples can be similar to the delivery shaft 54, is configured to be advanced through the patient's vasculature (blood vessel 12) and to the implantation site (such as native mitral valve 16) by the user and may be configured to retain the docking device 152 in a distal end portion 153 of the delivery shaft 154. During the docking device implantation procedure, the delivery shaft 154 is advanced through the catheter shaft 34 of the guide catheter 30 (e.g., through a central lumen thereof, etc.) and to the target implantation site.

The handle 156, which in some examples can be similar to the handle 56, is configured to be gripped and/or otherwise held by the user, outside the body of the patient 10, to advance the delivery shaft 154 through the patient's vasculature (such as the blood vessel 12). In some examples, the handle 156 can comprise one or more articulation members 157 (such as rotatable knobs) that are configured to aid in navigating the delivery shaft 154 through the blood vessel 12 by steering or controlling the flexing of the delivery apparatus 150 (e.g., the delivery shaft 154, etc.). Some examples of the articulation members 157 can be similar to the articulation members 57. The handle 156 comprises a handle lumen (not shown) extending through the length of the handle 156, wherein the handle lumen is configured to receive the sleeve shaft 182 and the pusher shaft 184. Since the sleeve and pusher shafts 182, 184 extending through the handle lumen also extend through the delivery shaft 154, the handle lumen is coaxially aligned with the delivery shaft 154. In some examples, the handle 156 can further comprise a locking assembly 198 configured to lock a device (for example, the sleeve shaft 182) inserted through the handle lumen, such that the device is selectively prevented from moving relative to the delivery apparatus 150. In some examples, the locking assembly 198 can be disposed on a proximal end portion of the handle 156.

The sleeve handle 196 is configured to be gripped and/or otherwise held by the user, outside the body of the patient 10, to advance the sleeve shaft 182 through the patient's vasculature. The sleeve handle 196 is coupled to a proximal end portion of the sleeve shaft 182 and is disposed proximal to the handle 156 and the hub assembly 200. The axial position of the sleeve shaft 182 can be controlled by moving the sleeve handle 196 in the axial direction relative to the handle 156 and/or the hub assembly 200.

The hub assembly 200 is configured to be gripped and/or otherwise held by the user, outside the body of the patient 10, to advance the pusher shaft 184 through the patient's vasculature. A proximal end portion of the pusher shaft 184 is coupled to the hub assembly 200 and can extend at least partially into the hub assembly 200. The hub assembly 200 is disposed axially between the handle 156 and the sleeve handle 196. In some instances, the axial position of the pusher shaft 184 can be controlled by moving the entire hub assembly 200 in the axial direction relative to the handle 156 and/or the sleeve handle 196.

Further details on delivery apparatus/catheters/systems (including various examples of the handle assembly) that are configured to deliver a docking device to a target implantation site can be found in PCT Publication Nos. WO 2020/247907 and WO 2022/072509, and U.S. Pat. Nos. 10,940,000 and 11,065,111, which are all incorporated by reference herein in their entireties.

The user of the docking device delivery apparatus 150 can perform the variable encircling turn (FIGS. 7-8) by moving the pusher shaft 184 in the axial direction relative to the delivery shaft 154 and the sleeve shaft 182. Since the delivery shaft 154 is coupled to the handle 156, the pusher shaft 184 is coupled to the hub assembly 200, and the sleeve shaft 182 is coupled to the sleeve handle 196, the variable encircling turn can be performed by moving the entire hub assembly 200 in the distal direction relative to the sleeve handle 196 while the handle 156 and the sleeve handle 196 are kept stationary.

The hub assembly 200 of the present disclosure can be configured to advance the pusher shaft 184 through the patient's vasculature, without requiring movement of the hub assembly 200 relative to the sleeve shaft 182. As described in more detail below, the hub assembly 200 can include a linear actuator 202 configured to advance the pusher shaft 184 relative to the hub assembly 200 (and relative to the sleeve shaft 182) to perform the variable encircling turn (FIGS. 7-8), if desired. The linear actuator 202 can, among other things, allow a user to finely adjust the relative positions of the pusher shaft (and thus the coil) and the sleeve shaft.

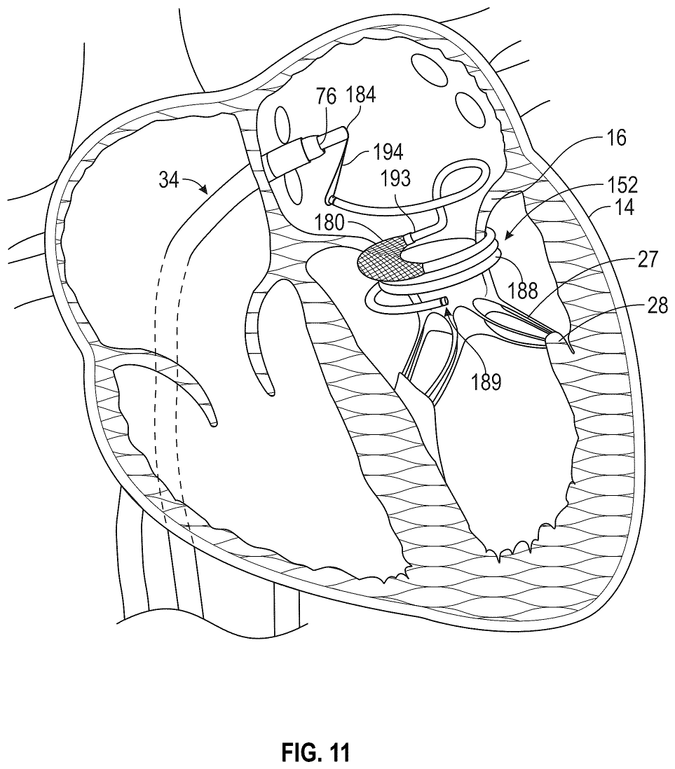

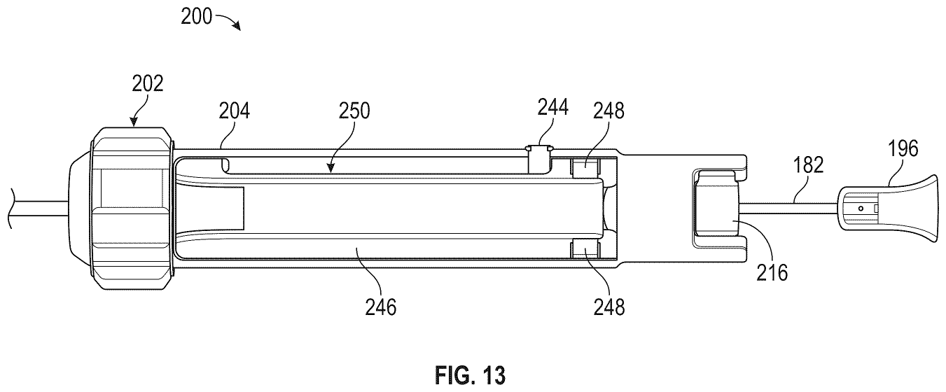

The hub assembly 200 includes an outer housing 204 and the linear actuator 202 can be at least partially disposed within the housing 204. For example, as shown in FIGS. 13-16, the linear actuator 202 can comprise an articulation member 206 (e.g., rotatable knob) coupled to the housing 204 and a traveler 208 (e.g., lead screw) disposed within the housing 204. The knob 206 is operatively coupled to the traveler 208, such that actuation of the knob 206 (e.g., rotation relative to the housing 204) results in translation of the traveler 208 relative to the housing 204. For example, the knob 206 can have a threaded inner surface 210 and the traveler 208 can have a threaded outer surface 212 that is operatively coupled to the threaded inner surface 210 (e.g., a threaded connection). As such, actuation (e.g., rotation) of the knob 206 can drive linear motion of the traveler 208 via the threaded connection between the surfaces 210, 212.

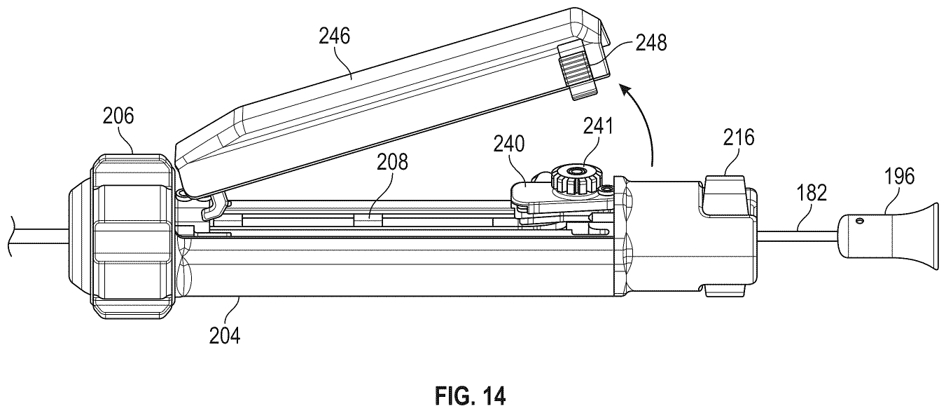

As described above, the sleeve shaft 182 extends through the hub assembly 200 to the sleeve handle 196. In particular, the sleeve shaft 182 extends through a hub assembly lumen 214 extending through the length of the hub assembly 200 (FIG. 16). The sleeve shaft 182 extends proximally from the hub assembly 200 and the sleeve handle 196 is positioned at the proximal end of the sleeve handle 196. The sleeve shaft 182 is configured to move (e.g., translate) relative to the housing 204 within the hub assembly lumen 214, for example, by moving the sleeve handle 196 relative to the hub assembly 200.

The pusher shaft 184 is disposed at least partially within the sleeve shaft 182 and extends partially into the hub assembly 200. The pusher shaft 184 is configured to move (e.g., translate) within the sleeve shaft 182 relative to the hub assembly housing 204 and/or relative to the sleeve shaft 182. The pusher shaft 184 is coupled to the traveler 208, such that the pusher shaft 184 and the traveler 208 are configured to translate axially together. In this way, operation of the linear actuator 202 causes movement (e.g., translation) of the pusher shaft 184 relative to the hub assembly 200.

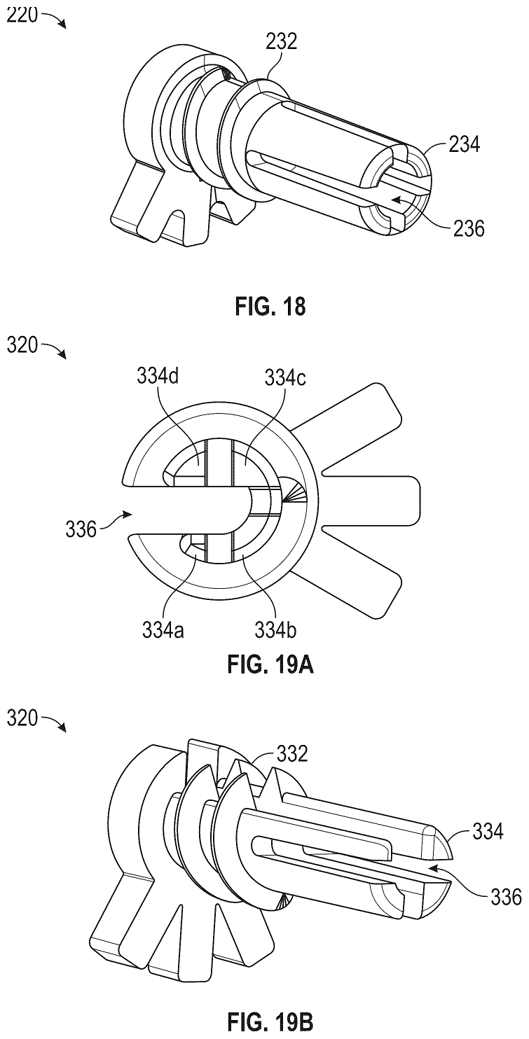

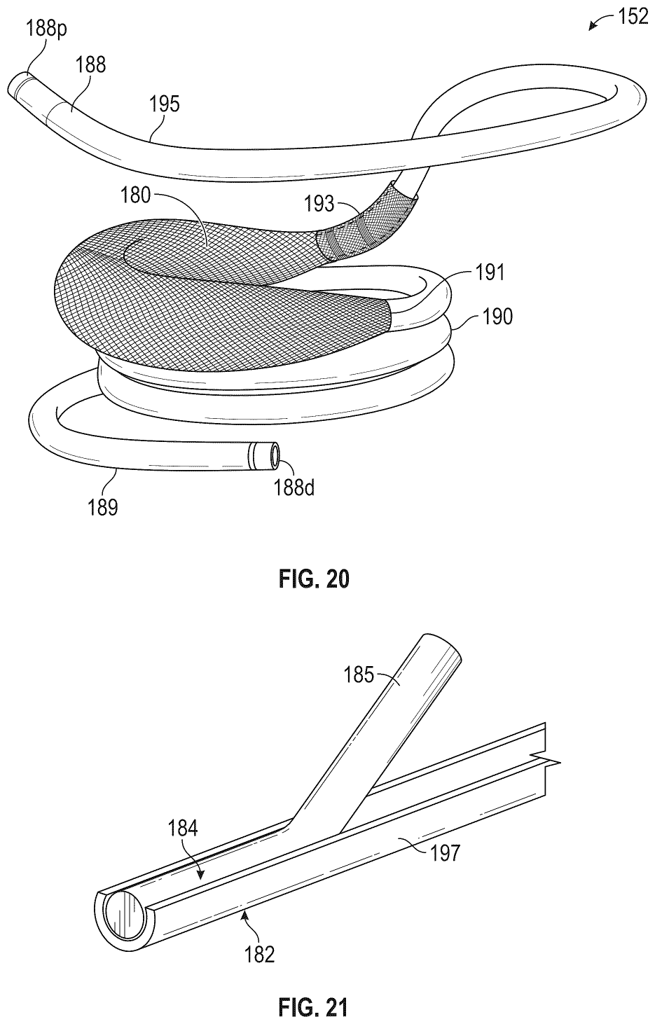

As described above, the pusher shaft 184 exits the sleeve shaft 182 at a location that is distal to the proximal end of the sleeve shaft 182 (e.g., proximal to the sleeve handle 196). For example, as shown in FIG. 21, a proximal end portion of the sleeve shaft 182 can have a partially annular (e.g., substantially U-shaped or C-shaped, etc.) axial cross-section that allows a proximal segment 185 of the pusher shaft 184 to exit the sleeve shaft 182 at an angle, relative to the sleeve shaft 182 (e.g., branched away from the sleeve shaft 182). The hub assembly 200 can be adapted and configured to allow the proximal segment 185 of the pusher shaft 184 to terminate within an interior region of the housing 204 (e.g., at an end of the traveler 208), while a proximal portion of the sleeve shaft 182 extends to the sleeve handle 196, disposed proximal to and external to the housing 204. With this configuration, a medical professional can execute the deployment of the docking device (e.g., docking device 152 of FIG. 20) by manipulating the position of the hub assembly 200 (e.g., moving the hub assembly 200 in the axial direction) and/or the traveler 208 (e.g., rotating the knob 206 relative to the housing 204) and also execute retraction of the sleeve shaft 182 off of and away from the implanted docking device by pulling back, in the axial direction, on the sleeve handle 196.

In this way, the sleeve shaft 182 and pusher shaft 184 can be configured to work together such that they can be moved simultaneously together when deploying and positioning the docking device at the native valve (e.g., by moving the entire hub assembly 200 forward and/or backward, in the axial direction), but can also to move independently, for example, to fine tune the positioning of the docking device at the native valve (e.g., by performing a VET as described above in connection with FIGS. 7-8) and/or to retract the sleeve shaft 182 off of the docking device while the pusher shaft 184 holds the docking device in position (e.g., by holding the hub assembly 200 in place relative to the delivery shaft 154 of the delivery apparatus 150 and/or other parts of the delivery apparatus 150 and/or docking device while pulling proximally on the sleeve handle 196 to withdraw the sleeve shaft 182).

As shown in FIG. 21, the proximal portion 197 of sleeve shaft 182 can define an open channel. For example, the channel of the sleeve shaft 182 is open in a radial direction, such that the proximal segment 185 of the pusher shaft 184 can extend out of the open channel and away from the sleeve shaft 182 at an angle relative to a longitudinal axis of the sleeve shaft 182. In some examples, the proximal portion 197 of the sleeve shaft 182 can also be referred to herein as an “open channel.” The open channel 197 can have a generally U or C-shaped cross-section. In some examples, as depicted, the open channel 197 has a curved outer surface, such that the open channel 197 has a partially annular cross-section (e.g., a C-shaped cross-section). Specifically, the open channel 197 can be partially annular with a concave, inwardly-facing surface and a convex outwardly-facing surface. In this way, the concave, inwardly-facing surface of the open channel 197 can form a void space in which the pusher shaft 184 can be at least partially disposed (FIG. 21). In various examples, the open channel 197 can be cut using a laser, although any other means for forming the open channel (e.g., removing part of the tubular structure) can be used.

A distal segment of the sleeve shaft 182 can comprise a closed channel or lumen, such that the channel is closed in the radial direction (e.g., an annular cross-section) (see FIG. 12). The pusher shaft 184 can extend through the closed channel of the sleeve shaft 182. For example, the pusher shaft 184 can be coaxial with the sleeve shaft 182 along some or a majority of the delivery apparatus 150, such as through the closed channel of the sleeve shaft 182. The open channel 197 can extend, for example, from an intermediate axial location of the sleeve shaft 182 to the proximal end of the sleeve shaft 182, for example, to the sleeve handle 196. In other examples, the open channel 197 of the sleeve shaft 182 can extend proximally from the intermediate axial location without extending to the proximal end of the sleeve shaft 182. In these examples, the open channel 197 can form an axially-extending window or slot that permits the proximal segment 185 of the pusher shaft 184 to extend out and way from the sleeve shaft 182 at an angle.

In some examples, because of frictional forces within the delivery apparatus 150, translation of the pusher shaft 184 based on operation of the linear actuator 202 may also cause translation of the sleeve shaft 182 within the hub assembly lumen 214. However, to perform the variable encircling turn (FIGS. 7-8), the pusher shaft 184 must move in the axial direction relative the sleeve shaft 182, rather than together with the sleeve shaft 182. To overcome frictional forces existing between the sleeve shaft 182 and the pusher shaft 184 when performing the variable encircling turn, the hub assembly 200 further includes a locking mechanism 216 in some examples. The locking mechanism 216 is configured to lock the sleeve shaft 182 relative to the hub assembly 200, such that the sleeve shaft 182 is selectively prevented from moving relative to the hub assembly 200. Thus, when the locking mechanism 216 is in a locked configuration, the linear actuator 202 can be operated to move the pusher shaft 184 in an axial direction while the locking mechanism 216 holds the sleeve shaft 182 stationary relative to the hub assembly 200. In this way, operation of the linear actuator 202 results in movement of the pusher shaft 184 relative to the hub assembly 200 (e.g., relative to the housing 204 and the sleeve shaft 182, etc.). In some examples, instead of a locking mechanism, a user of the delivery apparatus 150 can hold the sleeve shaft 182 stationary (e.g., by holding the sleeve handle 196) while operating the linear actuator 202, to perform the variable encircling turn.

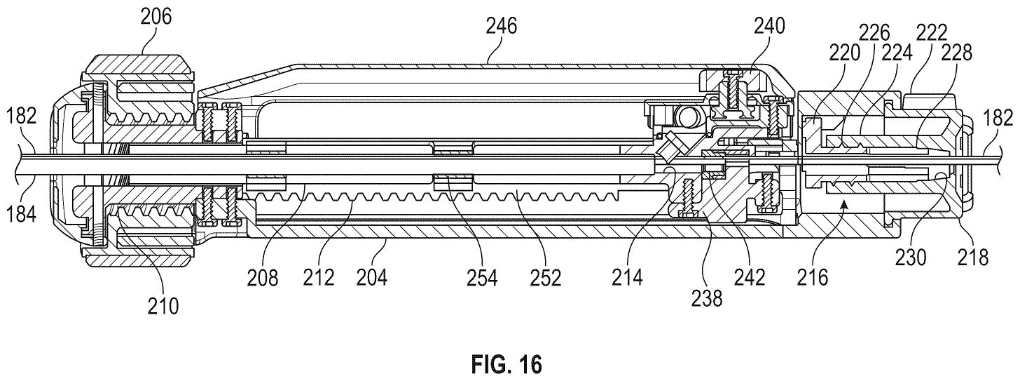

The locking mechanism 216 can be configured to prevent movement of the sleeve shaft 182 relative to the hub assembly 200 when the locking mechanism 216 is in the locked configuration. In the unlocked configuration, the locking mechanism 216 can be configured to allow such movement. Referring to FIGS. 16 and 18, the locking mechanism 216 can include a rotatable knob 218 (also referred to herein as a “locker body”) and a collet 220. The knob 218 includes two tabs 222 extending outwardly from the knob 218 in a radial direction and a shaft 224 extending distally from the knob 218 in an axial direction. The shaft 224 can be configured to receive the collet 220. In the illustrated example, the shaft 224 can include a threaded region 226 having internal threads and a tapered region 228. The tapered region 228 includes an inner surface 230 that can be tapered from a larger inner diameter to a smaller inner diameter in a proximal direction.

As shown in FIG. 18, the collet 220 can include external threads 232 configured to engage with the internal threads in the threaded region 226 of the shaft 224. The collet 220 can also include axially extending projections 234 (also referred to herein as “cantilevered arms”) at a proximal end of the collet 220. In some instances, as depicted, the collet 220 can include four projections 234. It should be appreciated that in other instances, the collet 220 can include a different number of projections 234. The collet 220 can also include a central lumen 236 extending from a distal end to the proximal end of the collet 220. The central lumen 236 can be coaxial with the hub assembly lumen 214 of the hub assembly 200.

When the locking mechanism 216 is in the unlocked configuration, the projections 234 extend straight out from the collet 220. In other words, the diameter of the central lumen 236 is uniform from the distal end to the proximal end of the collet 220. In the unlocked configuration, the diameter of the central lumen 236 can be the same as the diameter of the hub assembly lumen 214. The locking mechanism 216 can be configured such that rotating the knob 218 by a certain amount (e.g., a quarter turn, a half turn, one turn, multiple turns, etc.) with respect to the hub assembly 200 changes the locking mechanism 216 from the unlocked configuration to the locked configuration.

As the knob 218 is rotated, the collet 220 can move in an axial direction relative to the knob 218 towards the tapered region 228 of the shaft 224. When the locking mechanism 216 is in the locked configuration, the projections 234 can contact the inner surface 230 of the shaft 224 and can be pushed or flexed radially inwards by the taper of the inner surface 230. In other words, in the locked configuration, the diameter of the central lumen 236 is smaller at the proximal end of the collet 220 than at the distal end of the collet 220 due to the tapered inner surface 230. In this manner, the projections 234 can be configured to clamp around a device inserted through the hub assembly 200 (e.g., sleeve shaft 182) to lock the device in place. As such, the projections 234 can be configured to prevent movement of the device relative to the hub assembly 200 (e.g., relative to the housing 204, etc.).

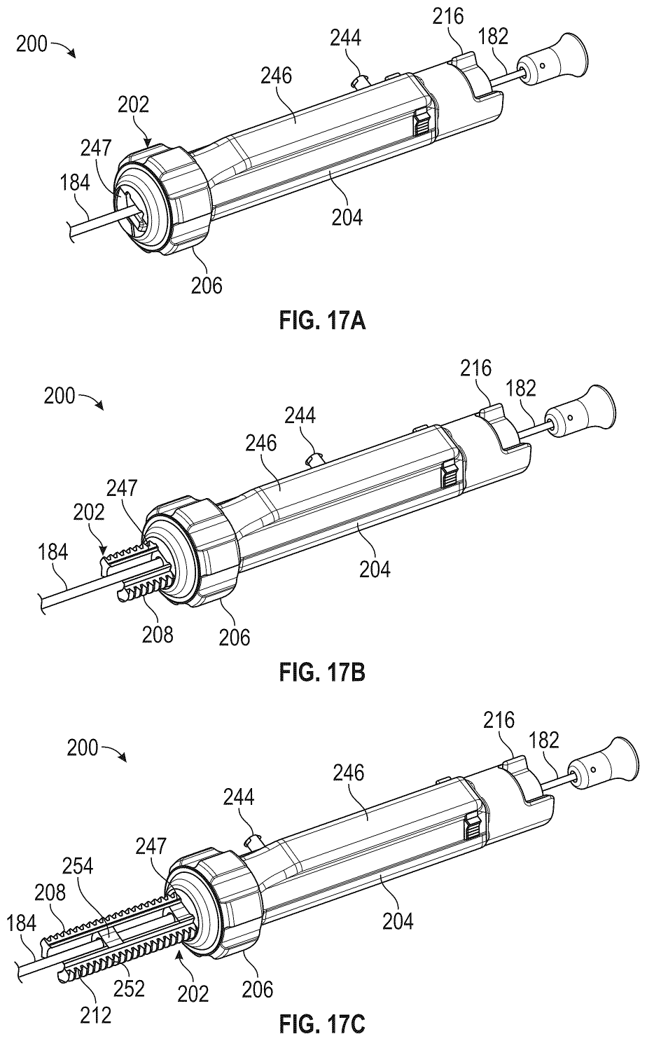

As shown in FIG. 18, the central lumen 236 of the collet 220 can have a circular cross-section, as defined by the projections 234. FIGS. 19A-B illustrate another example of a collet 320 that can be included in the locking mechanism 216, instead of collet 220. Collet 320 is similar to collet 220. For example, the collet 320 includes external threads 332 configured to engage with internal threads of the knob 218. However, a central lumen 336 of collet 320, as defined by extensions 334, has a different shaped cross-section than the central lumen 236 of the collet 220. As described above, the proximal portion 197 of the sleeve shaft 182 can have a partially annular cross-section (e.g., U-shaped cross-section, c-shaped cross-section, etc.) in some examples. To enable the collet 320 to engage with more surfaces of the partially annular sleeve shaft 182 and provide a sufficient locking force (e.g., sufficient to overcome frictional forces between the sleeve shaft 182 and the pusher shaft 184, etc.), the central lumen 336 of the collet 320 can be constructed to accommodate a partially-annular cross-section of the sleeve shaft 182. As shown, the extensions 334 can each comprise a different shape and/or size, with some extensions having a smaller cross-section (e.g., extension 334a) compared to the other extensions (e.g., extensions 334b-334d), with extension(s) having a curved inner surface (e.g., extension 334b), and/or a flat inner surface (e.g., extensions 334a, 334c, 334d). In some examples, as depicted in FIG. 19A, the central lumen 336 can be slotted from the distal to proximal ends of the collet 320, such that the sleeve shaft 182 can be inserted into the collet 320 in a radial direction, as well as in an axial direction. In some examples, the extensions 334 define a partially-annular shaped central lumen 336 that is not slotted. For example, the extensions 334 can define a central lumen 336 having a D-shaped cross-section to surround the partially annular cross-section of the sleeve shaft 182. The central lumen 336 can define other shapes that correspond to the cross-sectional shape of the shaft extending through the collet 320 (e.g., sleeve shaft 182).

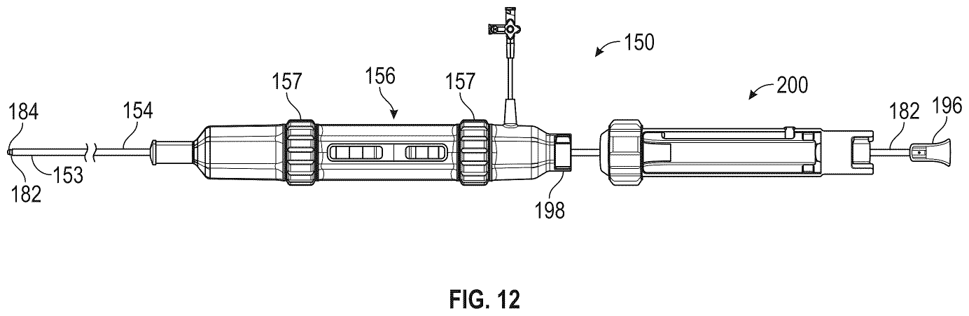

Referring to FIGS. 14-16, a chassis 238 (FIG. 16) can be coupled to a proximal end of the traveler 208 and configured to translate axially together with the traveler 208. A suture lock assembly 240 and one or more seals 242 can be coupled to the chassis 238. The hub assembly 200 can further include one or more flush ports (e.g., flush port 244) to supply flush fluid to one or more lumens arranged within the delivery apparatus 150 (e.g., annular lumens arranged between coaxial components of the delivery apparatus 150), for example, to maintain hemostasis within the delivery apparatus 150. The flush port 244 can be coupled to the chassis 238, for example, to supply flush fluid distal to the seals 242.

In some examples, the proximal end portion of the pusher shaft 184 can extend to the chassis 238 and be operatively coupled to the suture lock assembly 240. As described above, the proximal end portion of the pusher shaft 184 can be branched or angled away from the sleeve shaft 182, for example, to extend towards the suture lock assembly 240 in some examples. The suture lock assembly 240 is configured to releasably couple to a proximal end of the release suture 194. In some examples, the suture lock assembly 240 can include a rotator 241 (which also may be referred to as a “rotatable handle”) to increase and decrease tension on the release suture 194 which can extend from the suture lock assembly 240 through a lumen of the pusher shaft 184 to the docking device 152. The suture lock assembly 240 can be configured to cut the release suture 194 to release the docking device 152 from the delivery apparatus 150, for example, at the end of a procedure to implant the docking device 152. Further details of suture lock assemblies that can be used with hub assembly 200 are described in International Application Nos. PCT/US2023/025726 and PCT/US2023/025730, both of which are incorporated by reference herein in their entireties.

In some examples, a user may operate the suture lock assembly 240 at limited times during a procedure (e.g., at an end of the procedure to release the docking device 152 from the delivery apparatus 150). As such, it can be useful for the suture lock assembly 240 to be accessible to the user during those specific times but otherwise inaccessible to the user (e.g., to prevent inadvertent operation of the suture lock assembly 240). To ensure the suture lock assembly 240 is only accessible when the user intends to operate the suture lock assembly 240, the housing 204 of the hub assembly 200 can include a cap 246 (or cover) that is coupled to the rest of the housing 204 and at least partially removable from the housing 204. As shown in FIG. 14, the cap 246 can be partially removed from the housing 204 to expose an interior region of the housing 204 that houses the suture lock assembly 240. As such, with the interior region of the housing 204 exposed, a user can operate the suture lock assembly 240 and/or other components disposed within the housing 204. In some examples, the cap 246 can include tabs 248 that are operable to release the cap 246 from the rest of the housing 204. In some examples, as depicted, the cap 246 is hingedly coupled to the housing 204. For example, after a user depresses the tabs 248, a user can lift the cap 246 from the housing 204 such that the interior region is accessible to the user and the user can operate the suture lock assembly 240. In some examples, the cap 246 can be coupled to the housing 204 with a spring, such that the cap 246 is biased away from the housing 204 after the tabs 248 are depressed. In some examples, the cap 246 can be slidably attached to the housing 204, such that a user can slide the cap 246 relative to the housing 204 to expose the interior region within the housing 204. In some examples, the housing 204 does not include a cap 246, such that the suture lock assembly 240 is exposed to the user throughout the procedure.

As introduced above, one or more seals 242 may be disposed within the chassis 238, such that the chassis 238 at least partially defines a housing for the seals 242. The seals 242 are configured to seal around one or more shafts of the delivery apparatus 150 and provide hemostasis. For example, the seals 242 can be positioned at a proximal end of the chassis 238 and positioned around an outer surface of the sleeve shaft 182. As described above, the proximal portion 197 of the sleeve shaft 182 can have a partially annular cross-section and the seals 242 can be configured to seal around the partially annular cross-section. Further details of seals and seal assemblies for shafts are described in U.S. Provisional Patent Application No. 63/482,210, which is incorporated by reference herein in its entirety.

The cap 246 and/or the housing 204 can define a slot 250 between the cap 246 and the rest of the housing 204. The slot 250 extends axially along a length of the cap 246. As shown in FIG. 13, the slot 250 defines an opening into the housing 204 and the flush port 244 can extend outward from the chassis 238 and out of the housing 204 through the slot 250. As such, the flush port 244 can be accessible to a user when the cap 246 is closed, as well as when the user operates the linear actuator 202 to perform a variable encircling turn as described below.

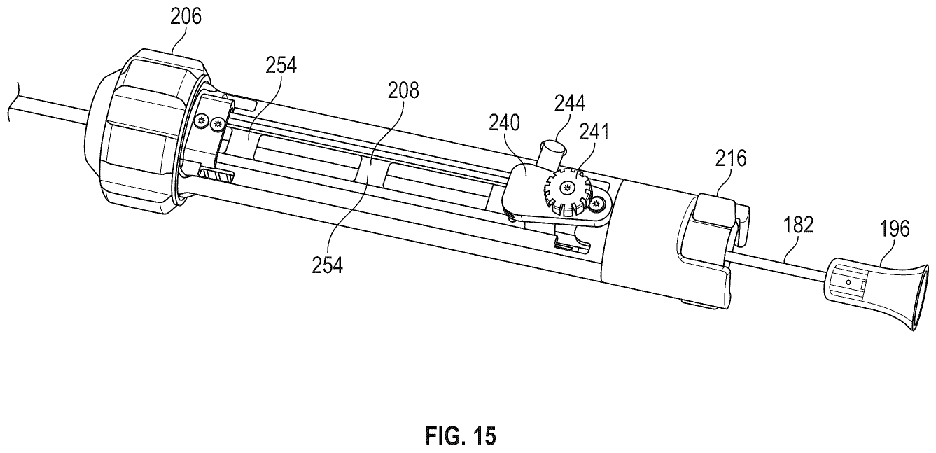

Referring to FIGS. 17A-17C, the axial position of the traveler 208 relative to the hub assembly 200 can be adjusted by operating the linear actuator 202 (e.g., by rotating knob 206). Because of the connection between the traveler 208 and the pusher shaft 184, translation of the traveler 208 results in translation of the pusher shaft 184. The locking mechanism 216 is illustrated in FIGS. 17A-17C in a locked configuration such that the sleeve shaft 182 is prevented from moving relative to the hub assembly 200. As described above, adjusting the axial position of the pusher shaft 184 relative to the sleeve shaft 182 can vary a magnitude of the radius of curvature of the sleeve shaft leading turn 187 (e.g., a VET as described above in connection with FIGS. 7-8). In particular, the radius of curvature of the sleeve shaft leading turn 187 correlates to the axial position of the pusher shaft 184 relative to the sleeve shaft 182.

In some examples, the traveler 208 can extend at least partially out of the housing 204 in some of axial positions to enable the traveler 208 to translate a greater axial distance without increasing the size of the hub assembly 200. For example, the traveler 208 can extend through an opening 247 at the distal end of the housing 204. In this way, the hub assembly 200 can be ergonomically sized for manipulation by a user while enabling a greater range of radius of curvature of the sleeve shaft leading turn 187. FIG. 17A illustrates the traveler 208 (and therefore the pusher shaft 184) in a first axial position relative to the hub assembly 200. In the first position, the traveler 208 is fully disposed within the housing 204. Rotating the knob 206 in a first direction relative to the housing 204 (e.g., clockwise) translates the traveler 208 and the pusher shaft 184 in a distal direction relative to the housing 204, for example, from the first axial position to a second axial position (FIG. 17B). In some examples, as depicted, a distal end of the traveler 208 can partially extend out of the housing 204 in the second axial position. When the traveler 208 is in the second axial position, rotating the knob 206 in the first direction relative to the housing 204 can further translate the traveler 208 and pusher shaft 184 in the distal direction relative to the housing 204 to a third axial position (FIG. 17C). In the third axial position, a larger portion of the traveler 208 extends out of the housing 204. Rotating the knob 206 in a second direction (e.g., counter-clockwise) can translate the traveler 208 and the pusher shaft 184 in a proximal direction relative to the housing 204, for example, from the third axial position to the second axial position. The first, second, and third axial positions each correspond to a different radius of curvature of the sleeve shaft leading turn 187. In this way, the radius of curvature of the sleeve shaft leading turn 187 can be adjusted by operating the linear actuator 202 to move the traveler 208 and the pusher shaft 184 between the axial positions shown in FIGS. 17A-17C. In other examples, the traveler 208 can be retained and enclosed within the housing 204 in all of the axial positions.

In some examples, the hub assembly 200 (e.g., the housing 204, the knob 206, and/or the traveler 208, etc.) can further comprise an indicator configured to indicate a magnitude of the variable encircling turn. Since the radius of curvature of the sleeve shaft leading turn 187 correlates to the axial position of the pusher shaft 184 relative to the sleeve shaft 182, and the axial position of the pusher shaft 184 relative to the sleeve shaft 182 correlates to the axial position of the housing 204 relative to the traveler 208, the radius of curvature of the sleeve shaft leading turn 187 can be determined based on the relative axial positions of the housing 204 and the traveler 208 and/or components coupled thereto (e.g., flush port 244). In some examples, the indicator can comprise one or more markings disposed along the length of the slot 250. A first marking disposed towards a proximal end of the slot 250 can indicate that the radius of curvature of the sleeve shaft leading turn 187 (in other words, the variable encircling turn) is equal to the first radius of curvature (r₁) and a second marking disposed towards a distal end of the slot 250 can indicate that the radius of curvature of the sleeve shaft leading turn 187 is equal to the second radius of curvature (r₂). In some examples, the indicator can be configured to indicate a magnitude of the variable encircling turn based on an amount of rotation of the knob 206 relative to the housing 204. The indicator can be a visual depiction of the coil (e.g., with various curvatures), words (e.g., “larger” and/or “smaller”), and/or any other indicia.

When the axial position of the traveler 208 is adjusted, the axial position of the components coupled to the traveler 208 is likewise adjusted. For example, the chassis 238, the suture lock assembly 240, the seals 242, and the flush port 244 can all translate together with the traveler 208. As described above, the flush port 244 can extend outward from the chassis 238 and out of the housing 204 through the slot 250. As such, the flush port 244 can translate along a length of the slot 250 when the linear actuator 202 is operated to move the traveler 208. In the first axial position (FIG. 17A), the flush port 244 is positioned adjacent to the proximal end of the hub assembly 200, for example, at a proximal end of the slot 250. In the third axial position (FIG. 17C), the flush port 244 is positioned adjacent to the distal end of the hub assembly 200, for example, at a distal end of the slot 250.

As shown in FIGS. 16 and 17C, the traveler 208 can comprise a pair of axially-extending rails 252 and a plurality of supports 254 extending between the rails 252. Each rail 252 includes the threaded outer surface 212 that can engage with the threaded inner surface 210 of the knob 206. The thread pitch of the threaded inner surface 210 can be varied to change the rate of linear actuation. For example, a smaller thread pitch of the threaded inner surface 210 results in slower actuation of the pusher shaft 184 relative to the housing 204, whereas a larger thread pitch will increase travel speed of the pusher shaft 184. In other words, for a given amount of rotation of the knob 206 relative to the housing 204 (e.g., a quarter turn, etc.), the traveler 208 can translate axially by different amounts based on the thread pitch of the threaded inner surface 210.

The sleeve shaft 182 and the pusher shaft 184 can extend through and/or between the supports 254 (e.g., through an opening of the supports 254). In some examples, the pusher shaft 184 can be fixedly coupled to the traveler 208 at one or more of the supports 254, such that the traveler 208 and the pusher shaft 184 move together in an axial direction. In some examples, the pusher shaft 184 can be fixedly coupled to the chassis 238. The sleeve shaft 182 can be configured to move relative to the traveler 208 (e.g., in an axial direction) through the openings defined by the supports 254.

FIG. 20 illustrates the docking device 152, according to one example. As depicted in FIG. 20, the docking device 152 in its deployed coiled configuration is configured to receive and secure a prosthetic valve (such as the prosthetic heart valve 62) within the docking device 152, thereby securing the prosthetic valve at the annulus of the native mitral valve 16.

The docking device 152 comprises a coil 188. In some examples, the coil 188 can include a shape memory material (e.g., nickel titanium alloy or “Nitinol”) such that the docking device 152 (and the coil 188) can move from a substantially straight configuration (or delivery configuration) when disposed within the delivery shaft 154 to a helical, deployed configuration after being removed from the delivery shaft 154.

The coil 188 has a proximal end 188p and a distal end 188d (which also respectively define the proximal and distal ends of the docking device 152). When being disposed within the delivery shaft 154 (for example, during delivery of the docking device 152 into the vasculature of a patient), a body of the coil 188 between the proximal end 188p and distal end 188d can form a generally straight delivery configuration (without any coiled or looped portions but can be flexed or bent) so as to maintain a small radial profile when moving through a patient's vasculature. After being removed from the delivery shaft 154 and deployed at an implant position, the coil 188 can move from the delivery configuration to the helical deployed configuration and wrap around native tissue adjacent the implant position. For example, when implanting the docking device at the location of a native valve, the coil 188 can be configured to surround native leaflets of the native valve (and the chordae tendineae that connects native leaflets to adjacent papillary muscles).

The coil 188 in the deployed coiled configuration can include the docking device leading turn 189, the central region 190, and a stabilization turn 195 (or “stabilization coil”) around a central longitudinal axis.

In the deployed coiled configuration, the central region 190 comprises one or more helical turns formed around a central longitudinal axis of the docking device 152, wherein the helical turns have substantially equal radii of curvature configured to encircle the leaflets 24 of the native mitral valve 16. The docking device leading turn 189 extends from a distal end of the central region 190 and has a radius of curvature greater than the radius of curvature of the helical turns of the central region 190. In some examples, the radius of curvature of the docking device leading turn 189 of the docking device 152 is equal to a second radius of curvature, wherein the second radius of curvature is less than the first radius of curvature of the sleeve shaft leading turn 187.

The stabilization turn 195 can extend from a proximal end of the central region 190 and has a diameter greater than the diameter of the central region 190, in the illustrated example. Alternatively, the stabilization turn 195 can have a diameter that is equal, approximately equal, or less than the diameter of the central region 190 (as opposed to larger), and/or the stabilization turn can comprise less of a full turn than depicted in FIG. 22.

In some examples, the docking device 152 can further comprise the guard member 180 disposed on the coil 188. The guard member is configured to reduce the possibility of paravalvular leakage between the native mitral valve 16 and the prosthetic heart valve. In some examples, the guard member 180 can comprise a braided portion disposed between the distal end portion 191 and the proximal end portion 193 of the guard member 180. The braided portion is configured to foreshorten into a deployed configuration when the proximal end portion 193 is forced in a distal direction, wherein the braided portion has an increased radial thickness in the foreshortened, deployed configuration.

Further details of the docking device and its variants are described in International Publication No. WO 2022/087336, which is incorporated by reference herein in its entirety.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for a docking device, the delivery apparatus comprising: a housing; a linear actuator coupled to the housing, the linear actuator comprising a traveler and an articulation member coupled to the traveler, wherein the traveler translates axially relative to the housing based on rotation of the articulation member relative to the housing; a first shaft extending through the housing and configured to translate axially relative to the housing; a second shaft extending through the first shaft and coupled to the traveler of the linear actuator such that the second shaft and the traveler translate axially together; and a locking mechanism coupled to the housing, wherein the first shaft is prevented from moving relative to the housing in a locked configuration, and wherein the first shaft is moveable relative to the housing in an unlocked configuration.

Example 2. The delivery apparatus of any example herein, particularly example 1, wherein a proximal portion of the first shaft comprises a partially annular axial cross-section.

Example 3. The delivery apparatus of any example herein, particularly either example 1 or example 2, wherein the locking mechanism includes a collet having a lumen, wherein the first shaft extends through the lumen.

Example 4. The delivery apparatus of any example herein, particularly example 3, wherein the lumen comprises a non-circular cross-section.

Example 5. The delivery apparatus of any example herein, particularly either example 3 or example 4, wherein the collet comprises extensions that are configured to clamp around the first shaft in the locked configuration, wherein the extensions are non-uniformly shaped and/or sized.

Example 6. The delivery apparatus of any example herein, particularly any one of examples 1-5, wherein the second shaft exits the first shaft distal to a proximal end of the first shaft.

Example 7. A delivery apparatus for a docking device, the delivery apparatus comprising: a housing including an opening at a distal end; a first shaft extending through the housing and configured to translate relative to the housing; a second shaft extending through the first shaft; and a linear actuator coupled to the housing, the linear actuator comprising a traveler and an articulation member coupled to the traveler, wherein the traveler translates axially relative to the housing between a first axial position and a second axial position based on rotation of the articulation member relative to the housing, wherein the second shaft and the traveler are configured to translate together, and wherein the traveler extends at least partially out of the opening of the housing in the second axial position.

Example 8. The delivery apparatus of any example herein, particularly example 7, wherein the traveler comprises a threaded outer surface, and wherein the articulation member comprises a threaded inner surface.

Example 9. The delivery apparatus of any example herein, particularly either example 7 or example 8, wherein the housing comprises a cap that is coupled to the housing and at least partially removable from the housing.

Example 10. The delivery apparatus of any example herein, particularly any one of examples 7-9, further comprising a locking mechanism coupled to the housing, wherein the first shaft is prevented from moving relative to the housing in a locked configuration, and wherein the first shaft is moveable relative to the housing in an unlocked configuration.

Example 11. The delivery apparatus of any example herein, particularly any one of examples 7-10, wherein the second shaft exits the first shaft distal to a proximal end of the first shaft.

Example 12. A delivery apparatus for a docking device, the delivery apparatus comprising: a housing defining an interior region; a linear actuator coupled to the housing and configured to translate axially relative to the housing, the linear actuator comprising a lead screw and a chassis coupled to the lead screw, the chassis positioned within the interior region of the housing; a pusher shaft extending through the housing and coupled to the lead screw, wherein the pusher shaft and the lead screw are configured to translate together relative to the housing; and a cap coupled to the housing and at least partially removable from the housing to selectively expose the interior region.

Example 13. The delivery apparatus of any example herein, particularly example 12, wherein the cap is rotatably connected to the housing.

Example 14. The delivery apparatus of any example herein, particularly example 12, wherein the cap is slidably connected to the housing.

Example 15. The delivery apparatus of any example herein, particularly any one of examples 12-14, further comprising a suture extending through the pusher shaft and a suture lock assembly coupled to the chassis, wherein the suture is releasably coupled to the suture lock assembly.

Example 16. The delivery apparatus of any example herein, particularly example 15, wherein the suture lock assembly is positioned within the interior region and accessible when the cap is partially removed from the housing.

Example 17. The delivery apparatus of any example herein, particularly example 16, wherein the housing and the cap define a slot extending axially along a length of the housing.

Example 18. The delivery apparatus of any example herein, particularly example 17, further comprising a flush port coupled to the chassis, wherein the flush port extends out of the interior region of the housing through the slot such that the flush port is accessible when the cap is closed onto the housing.

Example 19. The delivery apparatus of any example herein, particularly any one of examples 12-18, further comprising a sleeve shaft extending through the housing, wherein the pusher shaft extends through the sleeve shaft, and wherein the pusher shaft exits the sleeve shaft within the interior region of the housing.

Example 20. A delivery apparatus for a docking device, the delivery apparatus comprising: a housing; a sleeve shaft extending through the housing, wherein the sleeve shaft comprises a u-shaped or c-shaped axial cross section; and a locking mechanism coupled to the housing and comprising a collet having a lumen, wherein the sleeve shaft extends through the lumen, wherein the lumen comprises a non-circular cross-section, wherein the sleeve shaft is prevented from moving relative to the housing in a locked configuration, and wherein the sleeve shaft is moveable relative to the housing in an unlocked configuration.

Example 21. The delivery apparatus of any example herein, particularly example 20, wherein the collet comprises extensions that are configured to clamp around the sleeve shaft in the locked configuration, wherein the extensions are non-uniformly shaped and/or sized.

Example 22. The delivery apparatus of any example herein, particularly either example 20 or example 21, further comprising a pusher shaft extending through the sleeve shaft.

Example 23. The delivery apparatus of any example herein, particularly example 22, wherein the pusher shaft exits the sleeve shaft distal to a proximal end of the sleeve shaft.

Example 24. The delivery apparatus of any example herein, particularly either example 22 or example 23, further comprising a linear actuator coupled to the housing, the linear actuator comprising a traveler and an articulation member coupled to the traveler, wherein the traveler translates axially relative to the housing based on rotation of the articulation member relative to the housing, and wherein the pusher shaft is coupled to the traveler of the linear actuator such that the pusher shaft and the traveler translate axially together.

Example 25. The delivery apparatus of any example herein, particularly any one of examples 1-24, wherein the delivery apparatus is sterilized.

Example 26. A method for implanting a prosthetic medical device at a target implantation site in a subject, the method comprising: advancing a prosthetic medical device coupled to a distal end of a pusher shaft of a delivery apparatus and retained within a sleeve shaft of the delivery apparatus towards the target implantation site by moving the sleeve shaft and the pusher shaft in a distal direction relative to a handle of the delivery apparatus; locking a position of the sleeve shaft relative to a hub assembly of the delivery apparatus with a locking mechanism coupled to the hub assembly of the delivery apparatus; and actuating a linear actuator of the hub assembly to move the pusher shaft in an axial direction relative to the sleeve shaft and the hub assembly.

Example 27. The method of any example herein, particularly example 26, further comprising unlocking the sleeve shaft and moving the sleeve shaft in a proximal direction relative to the pusher shaft and the hub assembly to retract the sleeve shaft from the prosthetic medical device.

The features described herein with regard to any example can be combined with other features described in any one or more of the other examples, unless otherwise stated. For example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

What is claimed is:

1. A delivery apparatus for a docking device, the delivery apparatus comprising:
a housing;
a linear actuator coupled to the housing, the linear actuator comprising a traveler and an articulation member coupled to the traveler, wherein the traveler translates axially relative to the housing based on rotation of the articulation member relative to the housing;
a first shaft extending through the housing and configured to translate axially relative to the housing;
a second shaft extending through the first shaft and coupled to the traveler of the linear actuator such that the second shaft and the traveler translate axially together; and
a locking mechanism coupled to the housing, wherein the first shaft is prevented from moving relative to the housing in a locked configuration, and wherein the first shaft is moveable relative to the housing in an unlocked configuration.

2. The delivery apparatus of claim 1, wherein a proximal portion of the first shaft comprises a partially annular axial cross-section.

3. The delivery apparatus of claim 1, wherein the locking mechanism includes a collet having a lumen, wherein the first shaft extends through the lumen.

4. The delivery apparatus of claim 3, wherein the lumen comprises a non-circular cross-section.

5. The delivery apparatus of claim 3, wherein the collet comprises extensions that are configured to clamp around the first shaft in the locked configuration, wherein the extensions are non-uniformly shaped and/or sized.

6. The delivery apparatus of claim 1, wherein the second shaft exits the first shaft distal to a proximal end of the first shaft.

7. A delivery apparatus for a docking device, the delivery apparatus comprising:
a housing including an opening at a distal end;
a first shaft extending through the housing and configured to translate relative to the housing;
a second shaft extending through the first shaft; and
a linear actuator coupled to the housing, the linear actuator comprising a traveler and an articulation member coupled to the traveler, wherein the traveler translates axially relative to the housing between a first axial position and a second axial position based on rotation of the articulation member relative to the housing, wherein the second shaft and the traveler are configured to translate together, and wherein the traveler extends at least partially out of the opening of the housing in the second axial position.

8. The delivery apparatus of claim 7, wherein the traveler comprises a threaded outer surface, and wherein the articulation member comprises a threaded inner surface.

9. The delivery apparatus of claim 7, wherein the housing comprises a cap that is coupled to the housing and at least partially removable from the housing.

10. The delivery apparatus of claim 7, further comprising a locking mechanism coupled to the housing, wherein the first shaft is prevented from moving relative to the housing in a locked configuration, and wherein the first shaft is moveable relative to the housing in an unlocked configuration.

11. The delivery apparatus of claim 7, wherein the second shaft exits the first shaft distal to a proximal end of the first shaft.

12. A delivery apparatus for a docking device, the delivery apparatus comprising:
a housing defining an interior region;
a linear actuator coupled to the housing and configured to translate axially relative to the housing, the linear actuator comprising a lead screw and a chassis coupled to the lead screw, the chassis positioned within the interior region of the housing;
a pusher shaft extending through the housing and coupled to the lead screw, wherein the pusher shaft and the lead screw are configured to translate together relative to the housing; and
a cap coupled to the housing and at least partially removable from the housing to selectively expose the interior region.

13. The delivery apparatus of claim 12, wherein the cap is rotatably connected to the housing.

14. The delivery apparatus of claim 12, wherein the cap is slidably connected to the housing.

15. The delivery apparatus of claim 12, further comprising a suture extending through the pusher shaft and a suture lock assembly coupled to the chassis, wherein the suture is releasably coupled to the suture lock assembly.

16. The delivery apparatus of claim 15, wherein the suture lock assembly is positioned within the interior region and accessible when the cap is partially removed from the housing.

17. The delivery apparatus of claim 16, wherein the housing and the cap define a slot extending axially along a length of the housing.

18. The delivery apparatus of claim 17, further comprising a flush port coupled to the chassis, wherein the flush port extends out of the interior region of the housing through the slot such that the flush port is accessible when the cap is closed onto the housing.

19. The delivery apparatus of claim 12, further comprising a sleeve shaft extending through the housing, wherein the pusher shaft extends through the sleeve shaft, and wherein the pusher shaft exits the sleeve shaft within the interior region of the housing.
